(19) United States
(12) Patent Application Publication
(10) Pub. No.: US 2025/0277790 A1
(43) Pub. Date: Sep. 4, 2025

(54) QUANTIFICATION OF LIGAND BIAS AND MUTATION-INDUCED SIGNALING BIAS IN EGFR PHOSPHORYLATION IN DIRECT RESPONSE TO LIGAND BINDING

(71) Applicant: The Johns Hopkins University, Baltimore, MD (US)

(72) Inventors: Daniel WIRTH, Baltimore, MD (US); Kalina HRISTOVA, Baltimore, MD (US)

(21) Appl. No.: 19/053,030

(22) Filed: Feb. 13, 2025

Related U.S. Application Data
(60) Provisional application No. 63/552,734, filed on Feb. 13, 2024.

(51) Int. Cl. G01N 33/573

(52) U.S. Cl. CPC G01N 33/573; G01N 2333/705; G01N 2333/912; G01N 2440/14

(57) ABSTRACT

The present disclosures relate to novel methods for detecting and quantifying receptor tyrosine kinase phosphorylation in the absence of cytoplasmic molecules and without contributions from feedback loops and system bias; novel methods for screening for a ligand that induces preferential phosphorylation of a tyrosine residue on a receptor tyrosine kinase; novel methods for identifying a signaling bias induced by the receptor tyrosine kinase mutation; novel method for quantifying receptor tyrosine kinase phosphorylation upon ligand stimulation The methods can be used to determine a new intrinsic ligand bias, a new mutation-induced bias coefficient, and a transducer function describing RTK phosphorylation upon ligand stimulation. These critical descriptors of RTK activation are measured in direct response of the RTKs to ligand binding, without contributions from downstream signaling feedback loops and system bias.

### CROSS-REFERENCE TO RELATED APPLICATIONS

This The present application claims priority to U.S. Provisional Application No. 63/552,734, filed Feb. 13, 2024, which is incorporated herein by reference in its entirety.

### STATEMENT REGARDING FEDERALLY SPONSORED RESEARCH

This invention was made with government support under grant 2106031 awarded by the National Science Foundation and GM068619 awarded by the National Institutes of Health. The government has certain rights in the invention.

### SEQUENCE LISTING

The instant application contains a Sequence Listing which has been submitted electronically in XML file format and is hereby incorporated by reference in its entirety. The XML copy, created on Feb. 5, 2025, is named “JHU_42724_202_SequenceListing.xml” and is 2,908 bytes in size.

### FIELD

The present disclosures relate to novel methods for detecting and quantifying receptor tyrosine kinase phosphorylation in the absence of cytoplasmic molecules and without contributions from feedback loops and system bias; novel methods for screening for a ligand that induces preferential phosphorylation of a tyrosine residue on a receptor tyrosine kinase; novel methods for identifying a signaling bias induced by the receptor tyrosine kinase mutation; and novel method for quantifying receptor tyrosine kinase phosphorylation upon ligand stimulation.

### BACKGROUND

Receptor tyrosine kinases (RTKs) are single pass membrane receptors, which control cell growth, differentiation, motility, survival, and metabolism. They have been implicated in many diseases, and are valuable drug targets. RTKs transduce biochemical signals via lateral interactions in the plasma membrane, by forming catalytically active dimers. RTK dimerization, which is modulated by ligand binding, brings the kinase domains together in close proximity so they cross-phosphorylate each other on tyrosines in the activation loop. This activates the kinases and they phosphorylate additional tyrosines, which serve as binding sites for effector molecules, thus triggering downstream signaling cascades. Recent work has suggested that the signaling pathways originating from different RTK tyrosines can be differentially activated by different ligands—a phenomenon known as ligand bias.

Ligand bias has been studied primarily in the context of G-protein coupled receptors, where it has been shown to originate in the first step of signal transduction across the plasma membrane, i.e., due to differential signal propagation across the length of the receptor. For RTKs, the origin of ligand bias has been debated. Furthermore, experiments meant to explore RTK ligand bias have thus far probed for functional selectivity, which is “the combined effect of ligand and system bias.” While ligand bias is universal and pertains to all cell types as it depends on receptors and ligands, its manifestation may be different in different cells and tissues due to the system bias. System bias is determined by the cellular/tissue/physiological state context, including the identities of downstream signaling effectors in cells. For RTKs, it has been further shown that the abundances of signaling effectors can introduce system biases. (Salazar-Cavazos, E. et al. Multisite EGFR phosphorylation is regulated by adaptor protein abundances and dimer lifetimes. Mol. Biol. Cell 31, 695-708 (2020). Importantly, system bias can be perceived even at the level of RTK phosphorylation, which can be affected by feedback loops that operate within the cell. A recent review of the current state of the field emphasizes that “biased signaling represents very complex pharmacology, making experiment design, interpretation and description challenging and often inconsistent—causing confusion about what has really been measured and what can be concluded”. Kolb, P. et al. Community guidelines for GPCR ligand bias: IUPHAR review 32. Br. J. Pharmacol. 179, 3651-3674 (2022).

### SUMMARY

The presently disclosed subject matter generally relates to a method for detecting receptor tyrosine kinase phosphorylation. The method comprises culturing cells in a medium; transfecting the cells with nucleic acids encoding a receptor tyrosine kinase; inducing vesiculation using osmotic stress, wherein the cells release plasma membrane-derived vesicles; collecting the plasma membrane-derived vesicles; adding to one or more plasma membrane-derived vesicles: one or more ligands that bind to a receptor tyrosine kinase of a membrane of the plasma membrane-derived vesicle, ATP kinase, a phosphatase inhibitor, and one or more antibodies that recognize a phosphorylated tyrosine residue on the receptor tyrosine kinase in response to ligand stimulation, wherein each antibody comprises a fluorescent label; and detecting fluorescence of the fluorescent labels of the antibodies, thereby detecting phosphorylated tyrosine residues. Plasma membrane-derived vesicles are sub-membrane fragments shed from the plasma membrane of the cells. Advantageously, the plasma membrane-derived vesicles lack components of signaling feedback loops and permit diffusion of cytoplasmic molecules involved in downstream signaling; and thus phosphorylation of receptor tyrosine kinases occurs without contribution from RTK downregulation, feedback loops, and system bias. Moreover, the methods described herein advantageously utilize low sample volumes. The methods described herein also advantageously permit control of phosphorylation reactions. The phosphorylation reaction is initiated by adding ligands and an ATP kinase cocktail. Advantageously, soluble phosphatases are not present and membrane phosphatases are inhibited. Only mature receptor tyrosine kinases are present.

In some embodiments, the cells comprise Chinese hamster ovary (CHO) cells. In some embodiments, the cells do not exhibit endogenous EGFR. In some embodiments, the nucleic acids comprise plasmid DNA. In some embodiments, the nucleic acids are plasmid DNA encoding the extracellular and transmembrane domain (ECTM) of FGFR. In some embodiments, the nucleic acids are plasmid DNA encoding human epidermal growth factor receptor (EGFR). In some embodiments, the nucleic acids encode a FGF receptor, a EGF receptor, an insulin receptor, a PDGF receptor, a VEGF receptor, a FRF receptor, a CCK receptor, a NGF receptor, a HGF receptor, an Eph receptor, an AXL receptor, a TIE receptor, a RYK receptor, a DDR receptor, a RET receptor, a ROS receptor, a LTK receptor, a ROR receptor, a MuSK receptor, a LMR receptor, or a receptor of the RTK class XX; or a modified receptor tyrosine kinase.

In some embodiments, the ligands comprise EGF, TGFα, Epiregulin, or EGF-tetramethylrhodamine. In some embodiments, the ligands comprises a fluorescent label. In some embodiments, the fluorescent label comprises a fluorescent protein, a fluorescent protein derivative, or a modified fluorescent protein. In some embodiments, the fluorescent label comprises fluorescein isothiocyanate (FITC) or AlexaF488.

In some embodiments, the nucleic acids comprise a fluorescent label. In some embodiments, the fluorescent label comprises a fluorescent protein, a fluorescent protein derivative, or a modified fluorescent protein. In some embodiments, the fluorescent label is mTurquoise (mTurq). In some embodiments, the fluorescent label comprises a blue fluorescent protein (e.g. EBFP, EBFP2, Azurite, mKalama1), a cyan fluorescent protein (e.g. ECFP, Cerulean, CyPet, mTurquoise2), or a yellow fluorescent protein derivatives (e.g. YFP, Citrine, Venus, YPet), or a green fluorescent protein.

In some embodiments, the phosphatase inhibitor comprises sodium fluoride, sodium orthovanadate, beta-glycerophosphate, sodium pyrophosphate. In some embodiments, Mg²⁺ is added to one or more plasma membrane-derived vesicles. In some embodiments, ATP, MgCl₂, and sodium orthovanadate (Na₃ VO₄) added to one or more plasma membrane-derived vesicles. In some embodiments, the antibodies are anti-phosphotyrosine antibodies. In some embodiments, the antibodies have a hydrodynamic radii less than 4, 5, 6, 7, 8, 9, 10, 11 or 11.5 nm. In some embodiments, a first antibody type recognizes a first phosphorylated tyrosine residue on the receptor tyrosine kinase in response to ligand stimulation, and a second antibody type recognizes a second phosphorylated tyrosine residue on the receptor tyrosine kinase in response to ligand stimulation. In some embodiments, a third antibody type recognizes a third phosphorylated tyrosine residue on the receptor tyrosine kinase in response to ligand stimulation. In some embodiments, a fourth antibody type recognizes a fourth phosphorylated tyrosine residue on the receptor tyrosine kinase in response to ligand stimulation. In some embodiments, a fifth antibody type recognizes a fifth phosphorylated tyrosine residue on the receptor tyrosine kinase in response to ligand stimulation.

In some embodiments, after vesiculation, cytoplasmic signaling proteins diffuse through the membrane of the plasma membrane-derived vesicle, and bound cytoplasmic proteins disassociate from the membrane of the plasma membrane-derived vesicle. In some embodiments, after vesiculation, cytoplasmic signaling proteins that have a hydrodynamic radii less than 4, 5, 6, 7, 8, 9, 10, 11 or 11.5 nm diffuse through the membrane of the plasma membrane-derived vesicle. In some embodiments, after vesiculation, cytoplasmic signaling proteins that have a molecular weight less than 25, 30, 40, 50, 60, 70, 80, 90, 100, 110, 120, 130, 140, 150, 160, 170, 180, 190, 200, 210, 220, 230, 240, or 250 kDa and diffuse through the membrane of the plasma membrane-derived vesicle. In some embodiments, the cytoplasmic signaling proteins comprise the SH2 domain. In some embodiments, the cytoplasmic signaling proteins comprise Grb2-GFP, PLCγ-GFP, or other cytoplasmic proteins that bind to the receptor tyrosine kinase on a cytoplasmic side of the membrane. In some embodiments, the bound cytoplasmic proteins comprise PLCδ-PH-GFP or other cytoplasmic proteins are proteins that bind to lipids on a cytoplasmic side of the membrane.

In some embodiments, the method further comprises imaging of fluorescence intensity inside the plasma membrane-derived vesicles and fluorescence intensity outside of the plasma membrane derived-vesicles, wherein fluorescence intensity inside the plasma membrane-derived vesicles equals fluorescence intensity outside the plasma membrane-derived vesicles when phosphorylation comes to equilibrium and no feedback loop is present. In some embodiments, the method further comprises allowing phosphorylation of tyrosine residues on the receptor tyrosine kinase to reach equilibrium for at least 20, 30, 40, 50, or 60 minutes prior to fluorescence imaging. In some embodiments, the method further comprises quantifying phosphorylation of a tyrosine residue, wherein quantifying phosphorylation comprises imaging of fluorescence of fluorescent labels of the antibodies. In some embodiments, the method further comprises labeling a receptor tyrosine kinase of a plasma membrane-derived vesicle with a fluorescent label and quantifying a receptor tyrosine kinase concentration in the membrane of the plasma membrane-derived vesicle, wherein quantifying the receptor tyrosine kinase concentration comprises imaging of fluorescence of fluorescent labels of the receptor tyrosine kinases.

In some embodiments, the method further comprises screening for a ligand that induces preferential phosphorylation of one tyrosine residue on the receptor tyrosine kinase over phosphorylation of another tyrosine residue of the receptor tyrosine kinase. In some embodiments, two or more ligands that bind to a receptor tyrosine kinase are added to the one or more plasma membrane-derived vesicles; and two or more types of antibodies are added to the plasma membrane-derived vesicles, wherein a first antibody type recognizes a first phosphorylated tyrosine residue on the receptor tyrosine kinase in response to ligand stimulation and the first antibody types comprises a first fluorescent label, and wherein a second antibody type recognizes a second phosphorylated tyrosine residue on the receptor tyrosine kinase and the second antibody type comprises a second fluorescent label; and the method further comprises screening for a ligand that induces preferential phosphorylation of one tyrosine residue on the receptor tyrosine kinase over phosphorylation of another tyrosine residue of the receptor tyrosine kinase. Advantageously, the ligand bias screening methods described herein permit a determination of ligand bias on an absolute scale and free of system bias. The ligand bias screening methods described herein can be used to help screen for biased ligands that eliminate deleterious effects of pathogenic mutations.

In some embodiments, the method further comprises identifying a signaling bias induced by the receptor tyrosine kinase mutation. In some embodiments, some cells are transfected with nucleic acids encoding a wildtype receptor tyrosine kinase, and other cells are transfected with nucleic acids encoding a receptor tyrosine kinase comprising a mutation; two or more types of antibodies are added to the plasma membrane-derived vesicles collected from vesiculated cells transfected with nucleic acids encoding a wildtype receptor tyrosine kinase, wherein a first antibody type recognizes a first phosphorylated tyrosine residue on the wildtype receptor tyrosine kinase in response to ligand stimulation and the first antibody types comprises a first fluorescent label, and wherein a second antibody type recognizes a second phosphorylated tyrosine residue on the wildtype receptor tyrosine kinase and the second antibody type comprises a second fluorescent label; two or more types of antibodies are added to the plasma membrane-derived vesicles collected from vesiculated cells transfected with nucleic acids encoding a receptor tyrosine kinase comprising a pathogenic mutation, wherein a first antibody type recognizes a first phosphorylated tyrosine residue on the mutant receptor tyrosine kinase in response to ligand stimulation and the first antibody types comprises a first fluorescent label, and wherein a second antibody type recognizes a second phosphorylated tyrosine residue on the mutant receptor tyrosine kinase and the second antibody type comprises a second fluorescent label; and the method further comprises identifying a signaling bias induced by the receptor tyrosine kinase mutation, wherein identifying the signaling bias comprises comparing i) a ligand's induced preferential phosphorylation of one tyrosine residue of the wildtype receptor tyrosine kinase over phosphorylation of another tyrosine residue of the wildtype receptor tyrosine kinase and ii) the same ligand's induced preferential phosphorylation of one tyrosine residue of the mutant receptor tyrosine kinase over phosphorylation of another tyrosine residue of the mutant receptor tyrosine kinase. In some embodiments, the mutation is a pathogenic mutation. Receptor tyrosine kinases are critically important for human development, and are implicated in many growth disorders and cancers. Advantageously, the novel methods for identifying a mutation-induced signaling bias described herein allows quantification of how pathogenic mutations bias the first step in signal transduction across the plasma membrane, which is critical to finding cures.

In some embodiments, each ligand comprises a fluorescent label; and the method further comprises quantifying ligand binding, wherein quantifying ligand binding comprises imaging of fluorescence of fluorescent labels of ligands.

In some embodiments, the method further comprises quantifying receptor tyrosine kinase phosphorylation upon ligand stimulation for each plasma membrane-derived vesicle, wherein quantifying receptor tyrosine kinase phosphorylation upon ligand stimulation comprises quantifying ligand binding to the receptor tyrosine kinase and quantifying phosphorylation of a tyrosine residue simultaneously. In some embodiments, quantifying ligand binding to the receptor tyrosine kinase comprises imaging fluorescence of the fluorescent labels of the ligands on the membrane of a plasma membrane-derived vesicle, and quantifying phosphorylation of a tyrosine residue comprises imaging fluorescence of the fluorescent labels of the antibodies. Advantageously, the methods described herein informs whether an agonist can be improved further.

In some embodiments, the method further comprises quantifying a maximum phosphorylation per receptor in response to one ligand for a maximum of ligand-bound receptors per plasma membrane-derived vesicle. In some embodiments, the method further comprises labeling a receptor tyrosine kinase of a plasma membrane-derived vesicle with a fluorescent label and quantifying a receptor tyrosine kinase concentration in the membrane of the plasma membrane-derived vesicle, wherein quantifying the receptor tyrosine kinase concentration comprises imaging of fluorescence of fluorescent labels of the receptor tyrosine kinases; and quantifying a maximum phosphorylation per receptor in response to one ligand for a maximum of ligand-bound receptors per plasma membrane-derived vesicle. Advantageously, the methods described herein permit quantifying a maximum phosphorylation response of RTKs, which was previously unknown, and enables comparison of agonists on an absolute scale.

In some embodiments, the method further comprising quantifying recruitment of antibodies to the vesicle membrane, wherein quantifying recruitment of antibodies to the vesicle membrane comprises imaging of fluorescent membrane intensities.

In some embodiments, data processing of images is automated. In some embodiments data processing of images is automated using a neural network. The high-throughput methods described herein permit measurements of thousands of data points per dose response curve, and minimizes random errors due to white noise in imaging. Advantageously, the methods described herein can be used for high-throughput screening of biased inhibitors that eliminate deleterious effects of pathogenic RTK mutations.

Different embodiments, features and advantages of the presently disclosed subject matter will be more fully apparent from the disclosure below and appended claims.

### BRIEF DESCRIPTION OF THE DRAWINGS

Having thus described the presently disclosed subject matter in general terms, reference will now be made to the accompanying Figures, which are not necessarily drawn to scale, and wherein:

FIG. 1A-D shows plasma membrane-derived vesicles as a tool to probe RTK phosphorylation in response to ligand binding via imaging of fluorescent intensities of FITC-labeled dextrans inside and outside of the plasma membrane-derived vesicles (FIG. 1A) and the corresponding plot of FITC intensity inside/FITC intensity outside v. molecular weights of dextran-FITC (FIG. 1B); and imaging of fluorescence of FITC-labeled anti-pY 4G10 antibodies and igG-FITC isotype control antibodies (FIG. 1C) and the corresponding graph of fluorescence over time (min) (FIG. 1D).

FIG. 2A-C shows ligand bias for wildtype EGFR via raw dose response curves for Y1068 and Y1173 phosphorylation per EGFR molecule, for the ligands EGF, TGFα, and epiregulin (FIG. 2A); ligand bias plots depicting three comparisons for the three ligands (TGFα vs EGF (reference), epiregulin vs EGF (reference), and epiregulin vs TGFα (reference)) and the two tyrosine phosphorylation responses (Y1173 phosphorylation and y1068 phosphorylation) (FIG. 2B); and a table of three ligand bias coefficients for the three comparisons (TGFα vs EGF (reference), epiregulin vs EGF (reference), and epiregulin vs TGFα (reference)) using equation 1 (FIG. 2C).

FIG. 3A-C shows ligand bias in L834R EGFR phosphorylation via raw dose response curves for L834R EGFR in response to EGF, TGFα, and epiregulin (FIG. 3A); ligand bias plots depicting comparison for comparisons for the three ligands (TGFα vs EGF (reference), epiregulin vs EGF (reference), and epiregulin vs TGFα (reference)) and the two tyrosine phosphorylation responses (Y1173 phosphorylation and y1068 phosphorylation) (FIG. 3B); and a table of ligand bias coefficients for the three comparisons (TGFα vs EGF (reference), epiregulin vs EGF (reference), and epiregulin vs TGFα (reference)) using equation 1 (FIG. 3C).

FIG. 4A-D shows L834R mutation-induced signaling bias via L834R-induced bias plots in the presence of EGF, TGFα, and epiregulin (FIG. 4A-FIG. 4C); and a table of mutation-induced signaling bias coefficients that quantify the degree of signaling bias introduced by the mutation in EGFR signaling in response to a specific ligand; three comparisons (TGFα vs EGF (reference), epiregulin vs EGF (reference), and epiregulin vs TGFα (reference)) using equation 2 (FIG. 4D).

FIG. 5A-F shows rho-mEGF EGFR transducer function via fluorescence imaging of one vesicle imaged in three scans in three channels (EGF ligand, anti-pY antibody, and EGFR) at 5 nM rho-mEGF in the presence of ATP/kinase cocktail (FIG. 5A); ligand bias plots for rho-mEGFR and human EGF, TGFα, and epiregulin ligands and the two tyrosine phosphorylation responses (Y1173 phosphorylation and y1068 phosphorylation) (FIG. 5B); a plot representing the transducer function (phosphorylation response (fluorescence in the antibody channel divided by the fluorescence in the EGFR channel) as a function of the stimulus (ligand-bound EGFR fraction, f_{bound},) for each individual vesicle that was imaged in the three channels (FIG. 5C); a plot of fitted FIG. 5C data using equation 5 along with the data, binned in intervals of 0.1 on the x axis wherein only bins containing at least 50 vesicles are shown (FIG. 5D); a plot of the normalized transducer function, i.e., the dependence of Rₚₕₒₛₚₕₒ/Rₘₐₓ on the bound fraction, f_{bound} (FIG. 5E); and a table of absolute bias coeffecients calculated using equations 19-23 (FIG. 5F).

FIG. 6A-C shows neural network recognition and analysis of plasma-membrane derived vesicles with FGFR3-eYFP via: a) Typical result of the neural network analysis. The neural network is able to recognize good and bad vesicles of different intensities. b) Fluorescence intensity calculations for a single vesicle. Intensities per unit membrane area were obtained by integrating the Gaussian intensity profiles across the membrane, shown as a function of the distance from the vesicle center. Blue: experimentally measured profile. Red: fit to the experimentally measured profile, composed of a Gaussian intensity which is integrated and analyzed, and background. Yellow: residual between experimental profiles and fits. c) Comparison of histograms of FGFR3-eYFP concentrations from vesicles that are pre-selected by the neural network (blue) and the ones pre-selected manually by a trained researcher (green). The two analysis methods show good agreement.

FIG. 7 shows kinetics traces acquired for EGFR in response to TGF via a plot of intensity over time. FRET is measured concomitantly with measurements of EGFR and antibody concentrations on the membrane. The sensitized acceptor emission (grey) is calculated from the measured intensity in the FRET channel (black) after correcting for donor bleed-though and for direct acceptor emission. There is no sensitized acceptor emission and there is no decrease in donor fluorescence, indicative of zero FRET. Exemplary kinetic traces for one vesicle are shown.

FIG. 8A-D shows a schematic diagram showing two cases of bias (FIG. 8A and FIG. 8B) and two cases of no bias (FIG. 8C and FIG. 8D).

FIG. 9 shows the experimental set up to follow receptor phosphorylation in plasma membrane-derived vesicles which are permeant to macromolecules. RTKs in the vesicle membrane dimerize and get phosphorylated in response to ligand (blue star) binding. In the presence of ligand and ATP cocktail, a fluorescent specific anti-phosphoY antibody (green) is recruited to the membrane, and its recruitment is quantified, along with RTK concentrations in each vesicle.

FIG. 10 shows the thermodynamic cycle describing EGFR activation. * indicates that the dimeric receptors can be phosphorylated.

FIG. 11 shows concentrations of WT EGFR monomers and dimers, as a function of receptor and EGF concentrations. The monomer (green), unliganded dimer (yellow), liganded monomer (magenta), single-liganded dimer (orange), and double-liganded dimer (blue) distributions are plotted as a function of EGFR concentration (y-axis) and EGF concentration (x-axis) for every vesicle. The unliganded dimer concentrations (yellow) are used to correct the measured response in FIG. 2A and FIG. 3A. Data shown in this figure is derived from 986 vesicles in 3 independent experiments.

FIG. 12 shows WT and L834R EGFR phosphorylation in individual vesicles in response to EGF, TGFα, and epiregulin, after correction for basal phosphorylation. The red center bar represents the mean. Data shown in this figure were derived from 19579 vesicles in 48 independent experiments.

FIG. 13 shows Fitted corrected dose-response curves for WT and L834R EGFR, for the three ligands. Shown are mean and standard errors (if errors are not visible, they are smaller than the symbols). The solid lines are fits to all the single vesicle data in FIG. 2A and FIG. 3A (19579 vesicles in 48 independent experiments) using equation (1).

FIG. 14 shows a comparison of corrected fitted dose response curves for WT and L834R EGFR in response to EGF, TGFα, and epiregulin. Same curves as in FIG. 13, replotted to show the difference in phosphorylation between the wild-type and the mutant. Shown are means and standard errors (if errors are not visible, they are smaller than the symbols). The solid lines are fits to all the single vesicle data in FIG. 2A and FIG. 3A (19579 vesicles in 48 independent experiments) using equation (1).

FIG. 15 shows measured and corrected EGFR dose responses to rho-mEGF. Individual vesicle datapoints are shown (n=2173 for Y1068 and n=1987 for Y1173, 9 independent experiments). The red center bar represents the mean.

### BRIEF DESCRIPTION OF THE SEQUENCE LISTING

SEQ ID NO: 1 represents an oligonucleotide primer (5′-cccagcagtttggcccgcccaaaatctgtga-3′).

SEQ ID NO: 2 represents a oligonucleotide primer (5′-tcacagattttggggggcca aactgctggg-3′).

### DETAILED DESCRIPTION

The present disclosures relate to novel methods for detecting and quantifying receptor tyrosine kinase phosphorylation in the absence of cytoplasmic molecules and without contributions from feedback loops and system bias; novel methods for screening for a ligand that induces preferential phosphorylation of a tyrosine residue on a receptor tyrosine kinase; novel methods for identifying a signaling bias induced by the receptor tyrosine kinase mutation; novel method for quantifying receptor tyrosine kinase phosphorylation upon ligand stimulation These methods can be used to determine a new intrinsic ligand bias, a new mutation-induced signaling bias coefficient, and a transducer function describing RTK phosphorylation upon ligand stimulation. These critical descriptors of RTK activation are measured in direct response of the RTKs to ligand binding, without contributions from downstream signaling feedback loops and system bias. These method can be used for all RTKs; and can be automated for high-throughput screening of RTK inhibitors. For example, the described methods for identifying a signaling bias induced by a pathogenic receptor tyrosine kinase mutation can utilize automatic imaging and data processing, and can be used for high-throughput screening of biased inhibitors to eliminate the deleterious effects of pathogenic RTK mutations.

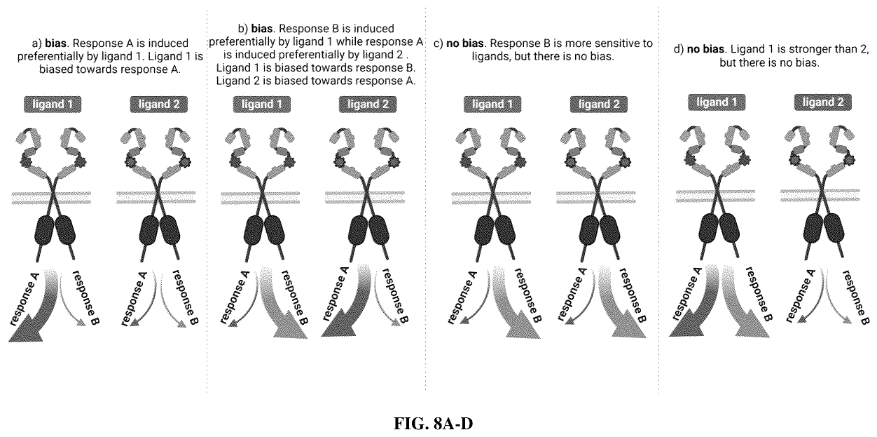

As defined herein, the term “ligand bias” describes the ability of ligands to preferentially activate a subset of signaling pathways, which can lead to fundamentally different biological responses (6, 7). Ligand bias does not simply reflect differences in potency (EC₅₀, the concentration of ligand that induces 50% of the maximum response), or efficacy (Eₜₒₚ, the maximum possible response due to a ligand). These differences represent quantitative differences in signaling, while bias represents fundamental (often called “qualitative”) differences in signaling (8-10). This is illustrated in FIG. 8 for two different ligands (blue and red) and two distinct responses (A, brown and B, green). Panels a and b show two cases of ligand bias while panels b and c show two cases of no bias. In FIG. 8A both ligands activate response B similarly, but ligand 1 induces response A more efficiently than ligand 2. In FIG. 8B ligand 1 preferentially induces response B while ligand 2 exhibits a preference towards response A. FIGS. 8B and 8C show only quantitative differences and therefore the ligands are not biased. FIG. 8B depicts the case where response B is more efficiently triggered than response A, but the two ligands act similarly without bias. In FIG. 8C ligand 1 activates both responses more efficiently than ligand 2, however the difference of activation in the two responses is the same for the two ligands and thus there is no bias. A variation of the last case would be where the efficiency of response B vary proportionally to the efficiency of response A. In such a case there would be no ligand bias, either. There have been numerous reports that propose the existence of ligand bias for FGF (13, 14), TRK (15-20), ERBB (21), Eph (22, 23), and PDGF (24) receptors. However, bias coefficients have not been calculated in these studies and the degree of the bias is unknown. Furthermore, it has been shown that different concentrations of adaptor proteins can bias signaling (25). This so-called “system bias” is often difficult to account for and decouple in cellular studies (26).

The concept of ligand bias complements and expands concepts in traditional pharmacology where ligands are classified as either an agonist, antagonist or inverse agonist (11). A biased ligand can be either an agonist, antagonist or inverse agonist (12).

As defined herein, mutation-induced signaling bias is defined in analogy to ligand bias, where instead of comparing the effects of different ligands, we compare the wild-type and the mutant in the presence of the same ligand. By calculating both β_{lig} and βₘᵤₜ from a comprehensive data set of dose-response curves, we uncouple and quantify biases introduced by ligand and by a pathogenic mutation.

Described herein are novel methodologies to:
identify and quantify intrinsic ligand bias in direct response to ligand binding, without contributions from feedback loops or system bias, and determine phosphorylation efficiencies and an absolute ligand bias coefficient;
identify and quantify signaling bias that is induced by an RTK mutation, and determine mutation-induced signaling bias coefficients; and
identify and quantify RTK phosphorylation upon ligand stimulation, and determine a transducer function.

The Examples show that the signaling of epidermal growth factor receptor (EGFR) to EGF and TGFα is biased towards Y1068 and against Y1173 phosphorylation, but has no bias for epiregulin. The Examples also that the L834R mutation found in non-small-cell lung cancer induces signaling bias as it switches the preferences to Y1173 phosphorylation. The knowledge gained here challenges the current understanding of EGFR signaling in health and disease and opens avenues for the exploration of biased inhibitors as anti-cancer therapies

The power of the methodology comes from the use of plasma derived vesicles produced via osmotic vesiculation. While these vesicles lack cytoskeleton and have perturbed asymmetry in their lipid composition, they allow access of macromolecules to both the extracellular and intracellular domains of the RTKs. In this respect, the plasma membrane-derived vesicles can be considered as an alternative to nanodisks. Unlike nanodisks, they do not impose artificial constraints on the free association of EGFR, and are a much more faithful mimic of the plasma membrane as they incorporate native lipids. They do not require RTK extraction out of the native plasma membrane and provide a contiguous membrane to ensure that the RTKs can associate with each other as they do in cells. Noteworthy, association constants measured for EGFR in vesicles and in cells are the same. Also noteworthy, plasma membrane-derived vesicles were recently leveraged in cryoEM studies to determine the high resolution structure of a membrane protein.

The use of vesicles allows us to make measurements of RTK phosphorylation in the absence of cytoplasmic molecules involved in downstream signaling and thus in the absence of system bias. The vesicles offer additional unique advantages. The phosphorylation reaction is initiated by the researcher, by adding ligand and ATP kinase cocktail, and phosphorylation is followed through the recruitment of labeled specific anti-pY antibodies to the vesicle membranes. There is no signal attenuation because there is no RTK downregulation. Soluble phosphatases are not present, and the membrane phosphatases are inhibited since the ATP kinase cocktail contains the inhibitors. Only mature RTKs in the plasma membrane are present. Antibodies, specific for only one tyrosine on only one RTK, verified in many RTK publications, are used in the detection. Data points in dose-response curves are derived from individual vesicles. Imaging is automated through the use of a commercial automated stage. Data processing is also automated using a neural network. The high-throughput format allows us to measure thousands of data points per dose response curve, and thus minimize random errors which arise due to white noise in imaging. Thus, the experimental platform is suitable for high-throughput screening of RTK inhibitors.

The data acquired with this method can be compared to published data. First, epiregulin is known to have lower potency for WT EGFR phosphorylation, as compared to EGF and TGFα11,34,37-39, in accordance with our measurements. Second, it is known that EGF and epiregulin signal differently through EGFR, since epiregulin induces cell differentiation under the same conditions where EGF induces proliferation. Ligand bias leads to fundamentally different biological outcomes, consistent with these prior findings and our observations of differential Y1068 and Y1173 phosphorylation. Thus, our results are consistent with knowledge in the literature. As an important development, we now construct bias plots, considered the most reliable proof of bias in the literature, and we calculate bias coefficients which support the bias plots. Thus, the degree of bias for multiple ligands is now quantified in the absence of feedback loops and system bias.

Another important result is the calculation of the EGF phosphorylation efficiency, which is the maximum possible phosphorylation that can be achieved in response to EGF. It is about 70% for Y1068 phosphorylation and 55% for Y1173 phosphorylation. This measurement is possible because ligand binding and EGFR phosphorylation are measured simultaneously, for hundreds of individual vesicles. The fit of the transducer function yields not only Kᵣₑₛₚ, used to calculate absolute bias coefficients, but also Rₘₐₓ, the maximum possible response to a ligand. We thus demonstrate that EGF is not a full agonist, which suggests that new ligands can be designed to more strongly activate EGFR.

We also gain insights into the origin of ligand bias in EGFR signaling. It has been argued that ligand bias in RTK signaling arises due to differential downregulation of the RTKs, or due to different abundances of cytoplasmic effectors. Here we show that bias arises in the first step of signal transduction, along the length of the RTK.

We introduce the concept of mutation-induced signaling bias coefficient, βₘᵤₜ, which reports on the preferences of pathogenic RTK mutants to differentially phosphorylate tyrosines as compared to the wild-type RTKs. Mutation-induced signaling bias is defined in analogy to ligand bias, where instead of comparing the effects of different ligands, we compare the wild-type and the mutant in the presence of the same ligand. By calculating both β_{lig} and βₘᵤₜ from a comprehensive data set of dose-response curves, we uncouple and quantify biases introduced by ligand and by a pathogenic mutation. By simultaneously measuring the ligand binding and phosphorylation for a fluorescently labeled ligand, we quantify the characteristics of the transducer function, which ultimately allows the calculation of absolute bias coefficients for natural ligands.

RTK mutations have been mainly classified as either gain of function (activating) or loss of function (deactivating) mutations. Here we show directly that the L834R EGFR mutation found in NSCLC induces bias in EGFR signal transduction across the plasma membrane. While EGFR signaling is biased toward Y1068 phosphorylation, the mutation switches the preference to Y1173 phosphorylation. It can be hypothesized that drug candidates that correct/unbias the first step in EGFR signal transduction can alter the signaling responses that are downstream from the mutant in a way that closely mimics WT EGFR signaling.

Our measurements set the stage for understanding how system bias modulates the effect of the L834R mutation on EGFR downstream signaling in physiological contexts. System bias acts in addition to ligand bias, and depends on the expression of downstream signaling molecules in the cells. Measurements of EGFR ligand and mutation-induced signaling bias in lung cancer cells will inform on the functional consequences of the differential signal transduction across the plasma membrane observed here. Studies can be expanded to investigate how the co-expression of WT EGFR and the L834R mutant affects signaling and cell physiology.

The demonstration of mutation-induced signaling bias will create an impetus to quantify mutation-induced signaling bias coefficients βₘᵤₜ for the many known RTK pathogenic mutations, and to reclassify the mutations based on the sign and magnitude of the bias coefficients. This will pave the way for the development of mutation-specific inhibitors which account for the discovered complexity in RTK signaling.

The presently disclosed subject matter generally relates to a method for detecting receptor tyrosine kinase phosphorylation. The method comprises culturing cells in a medium; transfecting the cells with nucleic acids encoding a receptor tyrosine kinase; inducing vesiculation using osmotic stress, wherein the cells release plasma membrane-derived vesicles; collecting the plasma membrane-derived vesicles; adding to one or more plasma membrane-derived vesicles: one or more ligands that bind to a receptor tyrosine kinase of a membrane of the plasma membrane-derived vesicle, ATP kinase, Mg²⁺, a phosphatase inhibitor, and one or more antibodies that recognize a phosphorylated tyrosine residue on the receptor tyrosine kinase in response to ligand stimulation, wherein each antibody comprises a fluorescent label; and detecting fluorescence of the fluorescent labels of the antibodies, thereby detecting phosphorylated tyrosine residues.

In some embodiments, the cells comprise Chinese hamster ovary (CHO) cells. In some embodiments, the cells do not exhibit endogenous EGFR. In some embodiments, the nucleic acids comprise plasmid DNA. In some embodiments, the nucleic acids are plasmid DNA encoding the extracellular and transmembrane domain (ECTM) of FGFR. In some embodiments, the nucleic acids are plasmid DNA encoding human epidermal growth factor receptor (EGFR). In some embodiments, the nucleic acids encode a FGF receptor, a EGF receptor, an insulin receptor, a PDGF receptor, a VEGF receptor, a FRF receptor, a CCK receptor, a NGF receptor, a HGF receptor, an Eph receptor, an AXL receptor, a TIE receptor, a RYK receptor, a DDR receptor, a RET receptor, a ROS receptor, a LTK receptor, a ROR receptor, a MuSK receptor, a LMR receptor, or a receptor of the RTK class XX; or a modified receptor tyrosine kinase.

In some embodiments, the ligands comprise EGF, TGFα, Epiregulin, or EGF-tetramethylrhodamine. In some embodiments, the ligands comprises a fluorescent label. In some embodiments, the fluorescent label comprises a fluorescent protein, a fluorescent protein derivative, or a modified fluorescent protein. In some embodiments, the fluorescent label comprises fluorescein isothiocyanate (FITC) or AlexaF488.

In some embodiments, the nucleic acids comprise a fluorescent label. In some embodiments, the fluorescent label comprises a fluorescent protein, a fluorescent protein derivative, or a modified fluorescent protein. In some embodiments, the fluorescent label is mTurquoise (mTurq). In some embodiments, the fluorescent label comprises a blue fluorescent protein (e.g. EBFP, EBFP2, Azurite, mKalama1), a cyan fluorescent protein (e.g. ECFP, Cerulean, CyPet, mTurquoise2), or a yellow fluorescent protein derivatives (e.g. YFP, Citrine, Venus, YPet), or a green fluorescent protein.

In some embodiments, the phosphatase inhibitor comprises sodium fluoride, sodium orthovanadate, beta-glycerophosphate, sodium pyrophosphate. In some embodiments, Mg²⁺ is added to one or more plasma membrane-derived vesicles. In some embodiments, ATP, MgCl₂, and sodium orthovanadate (Na₃ VO₄) added to one or more plasma membrane-derived vesicles. In some embodiments, the antibodies are anti-phosphotyrosine antibodies. In some embodiments, the antibodies have a hydrodynamic radii less than 4, 5, 6, 7, 8, 9, 10, 11 or 11.5 nm. In some embodiments, a first antibody type recognizes a first phosphorylated tyrosine residue on the receptor tyrosine kinase in response to ligand stimulation, and a second antibody type recognizes a second phosphorylated tyrosine residue on the receptor tyrosine kinase in response to ligand stimulation. In some embodiments, a third antibody type recognizes a third phosphorylated tyrosine residue on the receptor tyrosine kinase in response to ligand stimulation. In some embodiments, a fourth antibody type recognizes a fourth phosphorylated tyrosine residue on the receptor tyrosine kinase in response to ligand stimulation. In some embodiments, a fifth antibody type recognizes a fifth phosphorylated tyrosine residue on the receptor tyrosine kinase in response to ligand stimulation.

In some embodiments, after vesiculation, cytoplasmic signaling proteins diffuse through the membrane of the plasma membrane-derived vesicle, and bound cytoplasmic proteins disassociate from the membrane of the plasma membrane-derived vesicle. In some embodiments, after vesiculation, cytoplasmic signaling proteins that have a hydrodynamic radii less than 4, 5, 6, 7, 8, 9, 10, 11 or 11.5 nm diffuse through the membrane of the plasma membrane-derived vesicle. In some embodiments, after vesiculation, cytoplasmic signaling proteins that have a molecular weight less than 25, 30, 40, 50, 60, 70, 80, 90, 100, 110, 120, 130, 140, 150, 160, 170, 180, 190, 200, 210, 220, 230, 240, or 250 kDa and diffuse through the membrane of the plasma membrane-derived vesicle. In some embodiments, the cytoplasmic signaling proteins comprise the SH2 domain. In some embodiments, the cytoplasmic signaling proteins comprise Grb2-GFP, PLCγ-GFP, or other cytoplasmic proteins that bind to the receptor tyrosine kinase on a cytoplasmic side of the membrane. In some embodiments, the bound cytoplasmic proteins comprise PLCδ-PH-GFP or other cytoplasmic proteins are proteins that bind to lipids on a cytoplasmic side of the membrane.

In some embodiments, the method further comprises imaging of fluorescence intensity inside the plasma membrane-derived vesicles and fluorescence intensity outside of the plasma membrane derived-vesicles, wherein fluorescence intensity inside the plasma membrane-derived vesicles equals fluorescence intensity outside the plasma membrane-derived vesicles when phosphorylation comes to equilibrium and no feedback loop is present. In some embodiments, the method further comprises allowing phosphorylation of tyrosine residues on the receptor tyrosine kinase to reach equilibrium for at least 20, 30, 40, 50, or 60 minutes prior to fluorescence imaging. In some embodiments, the method further comprises quantifying phosphorylation of a tyrosine residue, wherein quantifying phosphorylation comprises imaging of fluorescence of fluorescent labels of the antibodies. In some embodiments, the method further comprises labeling a receptor tyrosine kinase of a plasma membrane-derived vesicle with a fluorescent label and quantifying a receptor tyrosine kinase concentration in the membrane of the plasma membrane-derived vesicle, wherein quantifying the receptor tyrosine kinase concentration comprises imaging of fluorescence of fluorescent labels of the receptor tyrosine kinases.

In some embodiments, the method further comprises screening for a ligand that induces preferential phosphorylation of one tyrosine residue on the receptor tyrosine kinase over phosphorylation of another tyrosine residue of the receptor tyrosine kinase. In some embodiments, two or more ligands that bind to a receptor tyrosine kinase are added to the one or more plasma membrane-derived vesicles; and two or more types of antibodies are added to the plasma membrane-derived vesicles, wherein a first antibody type recognizes a first phosphorylated tyrosine residue on the receptor tyrosine kinase in response to ligand stimulation and the first antibody types comprises a first fluorescent label, and wherein a second antibody type recognizes a second phosphorylated tyrosine residue on the receptor tyrosine kinase and the second antibody type comprises a second fluorescent label; and the method further comprises screening for a ligand that induces preferential phosphorylation of one tyrosine residue on the receptor tyrosine kinase over phosphorylation of another tyrosine residue of the receptor tyrosine kinase.

In some embodiments, the method further comprises identifying a signaling bias induced by the receptor tyrosine kinase mutation. In some embodiments, some cells are transfected with nucleic acids encoding a wildtype receptor tyrosine kinase, and other cells are transfected with nucleic acids encoding a receptor tyrosine kinase comprising a mutation; two or more types of antibodies are added to the plasma membrane-derived vesicles collected from vesiculated cells transfected with nucleic acids encoding a wildtype receptor tyrosine kinase, wherein a first antibody type recognizes a first phosphorylated tyrosine residue on the wildtype receptor tyrosine kinase in response to ligand stimulation and the first antibody types comprises a first fluorescent label, and wherein a second antibody type recognizes a second phosphorylated tyrosine residue on the wildtype receptor tyrosine kinase and the second antibody type comprises a second fluorescent label; two or more types of antibodies are added to the plasma membrane-derived vesicles collected from vesiculated cells transfected with nucleic acids encoding a receptor tyrosine kinase comprising a pathogenic mutation, wherein a first antibody type recognizes a first phosphorylated tyrosine residue on the mutant receptor tyrosine kinase in response to ligand stimulation and the first antibody types comprises a first fluorescent label, and wherein a second antibody type recognizes a second phosphorylated tyrosine residue on the mutant receptor tyrosine kinase and the second antibody type comprises a second fluorescent label; and the method further comprises identifying a signaling bias induced by the receptor tyrosine kinase mutation, wherein identifying the signaling bias comprises comparing i) a ligand's induced preferential phosphorylation of one tyrosine residue of the wildtype receptor tyrosine kinase over phosphorylation of another tyrosine residue of the wildtype receptor tyrosine kinase and ii) the same ligand's induced preferential phosphorylation of one tyrosine residue of the mutant receptor tyrosine kinase over phosphorylation of another tyrosine residue of the mutant receptor tyrosine kinase. In some embodiments, the mutation is a pathogenic mutation.

In some embodiments, each ligand comprises a fluorescent label; and the method further comprises quantifying ligand binding, wherein quantifying ligand binding comprises imaging of fluorescence of fluorescent labels of ligands.

In some embodiments, the method further comprises quantifying receptor tyrosine kinase phosphorylation upon ligand stimulation for each plasma membrane-derived vesicle, wherein quantifying receptor tyrosine kinase phosphorylation upon ligand stimulation comprises quantifying ligand binding to the receptor tyrosine kinase and quantifying phosphorylation of a tyrosine residue simultaneously. In some embodiments, quantifying ligand binding to the receptor tyrosine kinase comprises imaging fluorescence of the fluorescent labels of the ligands on the membrane of a plasma membrane-derived vesicle, and quantifying phosphorylation of a tyrosine residue comprises imaging fluorescence of the fluorescent labels of the antibodies.

In some embodiments, the method further comprises quantifying a maximum phosphorylation per receptor in response to one ligand for a maximum of ligand-bound receptors per plasma membrane-derived vesicle. In some embodiments, the method further comprises labeling a receptor tyrosine kinase of a plasma membrane-derived vesicle with a fluorescent label and quantifying a receptor tyrosine kinase concentration in the membrane of the plasma membrane-derived vesicle, wherein quantifying the receptor tyrosine kinase concentration comprises imaging of fluorescence of fluorescent labels of the receptor tyrosine kinases; and quantifying a maximum phosphorylation per receptor in response to one ligand for a maximum of ligand-bound receptors per plasma membrane-derived vesicle.

In some embodiments, the method further comprising quantifying recruitment of antibodies to the vesicle membrane, wherein quantifying recruitment of antibodies to the vesicle membrane comprises imaging of fluorescent membrane intensities.

In some embodiments, the In some embodiments, data processing of images is automated. In some embodiments data processing of images is automated using a neural network.

The present subject matter may be a system, a method, and/or a computer program product. In some embodiments, the computer program product may include a computer readable storage medium (or media) having computer readable program instructions thereon for causing a processor to carry out aspects of the present subject matter.

In some embodiments, the computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a RAM, a ROM, an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

In some embodiments, computer readable program instructions described herein can be downloaded to respective computing/processing devices from a computer readable storage medium or to an external computer or external storage device via a network, for example, the Internet, a local area network, a wide area network and/or a wireless network, or Near Field Communication. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

In some embodiments, computer readable program instructions for carrying out operations of the present subject matter may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Smalltalk, C++, Javascript or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present subject matter.

In some embodiments, the computer readable program instructions may be provided to a processor of a computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. In some embodiments, the computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

In some embodiments, the computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The features and advantages of the presently disclosed subject matter will be understood more readily by reference to the examples discussed below, which are provided by way of illustration and are not intended to be limiting of the presently disclosed subject matter.

### Examples

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The descriptions and specific examples that follow are only intended for the purposes of illustration and are not to be construed as limiting in any manner to make compounds, vesicles, or compositions of the disclosure by other methods.

All single vesicle data generated in this study have been deposited in the figshare database under accession code: figshare.com/articles/dataset/Quantification_of_ligand_and_mutation-induced_bias_in_EGFR_phosphorylation_in_direct_response_to_ligand_binding/24162846 and incorporated herein by reference.

### Example 1: Novel Model System to Measure RTK Phosphorylation in Direct Response to Ligand Binding Using Plasma Membrane-Derived Vesicles Produced Via Osmotic Vesiculation

### Plasmid Constructs

The plasmid encoding for human EGFR, tagged with the fluorescent protein mTurquoise (mTurq) at the C-terminus via a flexible GGS linker, is in the pSSX vector. The L834R mutation was introduced in EGFR using the QuikChange II Site-Directed Mutagenesis Kit according to manufacturer's instructions (Agilent Technologies, #200523). Primers: 5′-cccagcagtttggcccgcccaaaatctgtga-3′ (SEQ ID NO: 1) and 5′-tcacagattttggggggcca aactgctggg-3′ (SEQ ID NO: 2). The plasmid used for the neural network training encoded for the extracellular and transmembrane domain (ECTM) of FGFR, a (GGS) 5 linker, and mTurq in the pcDNA3.1 (+) vector. All plasmids were sequenced to confirm their identity (Genewiz).

### Cell Culture and Vesiculation

Chinese hamster ovary (CHO) cells were purchased from ATCC. CHO cells were used in these experiments as they do not exhibit endogenous EGFR expression. CHO cells were cultured in Dulbecco's modified Eagle medium (Gibco, #31600034) supplemented with 10% fetal bovine serum (HyClone, #SH30070.03), 1 mM nonessential amino acids, 10 mM D-glucose, and 18 mM sodium bicarbonate at 37° C. in a 5% CO2 environment. Cells were passed every other day using standard tissue culture techniques.

For vesiculation, the cells were seeded in a 6-well plate at a density of 2*10⁴ cells per well. 24 h later, the cells were transfected with 1 or 1.5 μg plasmid DNA using FuGene HD (Promega, #E2311) according to the manufacturer's protocol. 36 h after transfection, vesiculation was induced using osmotic stress as described in Del Piccolo, N., Placone, J., He, L., Agudelo, S. C. & Hristova, K. Production of plasma membrane vesicles with chloride salts and their utility as a cell membrane mimetic for biophysical characterization of membrane protein interactions (Analy. Chem. 84, 8650-8655 (2012)), which is incorporated herein by reference.

The osmotic pressure stresses the cells such that they release vesicles into solution without causing substantial cell detachment. The vesicles that were released in solution were collected by aspirating the supernatant with a cut 1000 μl micropipette tip.

### Cho Vesicle Characterization Using Dextran Solutions

To characterize the permeability of CHO vesicles to macromolecules, FITC-labeled dextran was added to the vesicle solution and the ratio of FITC intensity inside and outside the vesicles was calculated for five different dextrans of sizes 20-2000 kDa. Dextrans were purchased from Sigma-Aldrich (#FD20S, #FD70S, #FD250S, #FD500S, and #FD2000S). After vesiculation, CHO vesicles with ECTM FGFR3 tagged with mTurq incorporated in the plasma membrane were transferred to an 8-well glass bottom chamber slide (ibidi, #80827). 100 nM of FITC-labeled dextran in osmotic chloride salt buffer was added to the vesicles. The chamber slide was transferred to a TCS SP8 confocal microscope (Leica Biosystems, Wetzlar, Germany) equipped with an automated stage and a HyD hybrid detector in photon counting mode. The vesicles were allowed to settle for 1 h. Image acquisition was automated by selecting pre-defined regions and focus points in the LAS X Navigator software (Leica Biosystems, Wetzlar, Germany). Two scans (256×256 pixels) per image were acquired, a ‘mTurq’-scan (A=448 nm, emission window: 460-510 nm), where m Turq bound to FGFR3 is excited, and a ‘FITC’-scan (A=488 nm, emission window: 500-540 nm), where FITC bound to dextran is excited. Images were acquired at 1% laser power with a 100 Hz scanning speed. To analyze the images, we developed a neural network approach as described in Example 6

### A Model System to Measure RTK Phosphorylation in Direct Response to Ligand Binding

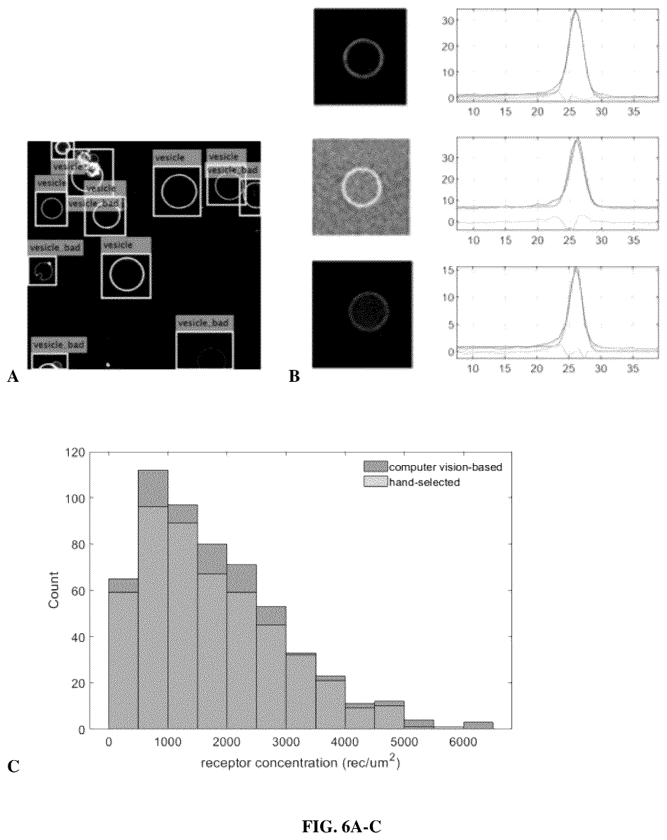

To be able to measure RTK phosphorylation in direct response to ligands without contribution from feedback loops and system bias, we used plasma membrane-derived vesicles produced via osmotic vesiculation. Such vesicles are produced from cells that have been transfected with genes encoding RTKs labeled with fluorescent proteins, and are imaged in a confocal microscope. As described in Example 6, we developed a neural network approach that allows high-throughput vesicle analysis. Once vesicles are identified, their membrane intensity is quantified as shown in FIG. 6. The fluorescence intensities inside and outside the vesicles are quantified as well.

While all plasma membrane-derived vesicles are known to have defects that allow the passage of macromolecules through the membrane, the vesicles produced via osmotic vesiculation allow the passage of very large macromolecules. As a result, cytoplasmic signaling proteins such as Grb2-GFP (MW=60 kDa) and PLCγ-GFP (MW=210 kDa) diffuse through the vesicle membranes and become infinitely diluted in the buffer which is contiguous with the vesicle lumens. So do cytoplasmic proteins that bind to the lipids on the cytoplasmic side (such as PLCδ-PH-GFP), or to RTKs on the cytoplasmic side (such as PLCγ-GFP). These proteins dissociate from the membrane because their residence times on the membrane are much shorter than the timescale of vesicle production, −12 h. Thus, these vesicles lack the components of signaling feedback loops.

Previous work has shown that the lipid composition of the vesicles is very similar to the lipid composition of the plasma membrane. Here we investigated the permeability of these vesicles to FITC-labeled dextrans (20-2000 kDa) which were added externally after vesicle production. The FITC intensity inside and outside the vesicles were measured after 1 h to quantify the degree of dextran penetration through the vesicle membrane.

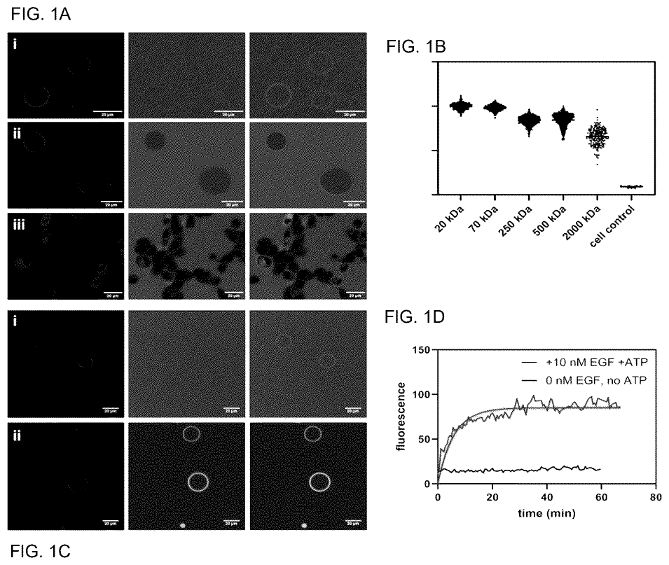

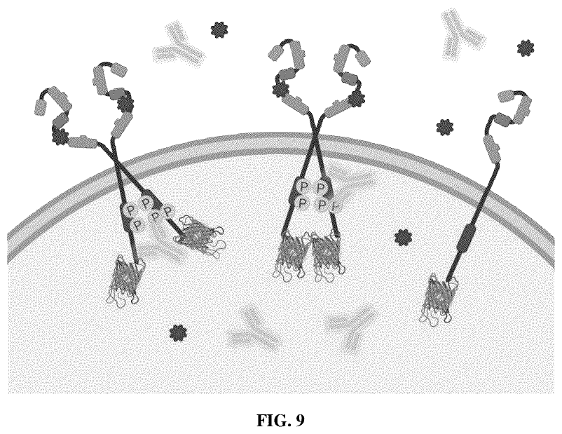

FIG. 1A and FIG. 1B show plasma membrane-derived vesicles as a tool to probe RTK phosphorylation in response to ligand binding via imaging of fluorescent intensities of FITC-labeled dextrans inside and outside of the plasma membrane-derived vesicles (1A) and the corresponding plot of FITC intensity inside/FITC intensity outside v. molecular weights of dextran-FITC (1B). FIG. 1A shows determination of the size cutoff for equilibration across the vesicle membrane. FITC-labeled dextrans of different molecular weights were added to vesicles with FGFR3-mTurquoise in their membrane. Left column: receptor, middle column: dextran, right column: overlay. (i) 70 kDa dextran. The intensity inside the vesicle and outside is the same. (ii) 2000 kDa dextran. The intensity inside the vesicle is lower. Shown are representative images from 11 independent vesicle experiments. (iii) cells, EGFR-mTurquoise+control antibody (rat IgG2bκ-FITC). The antibody does not cross the cell membrane. Shown are representative images from 3 independent cell experiments. FIG. 1B shows intensity ratios between FITC-labeled dextran inside and outside of vesicles for dextrans of different molecular weights. Each data point represents the ratio for one vesicle. Data are for 3454 vesicles in 14 independent experiments. FIG. 1A and FIG. 1B show that the 20 kDa and 70 kDa dextrans equilibrate across the vesicle membrane without any obstruction (intensity ratio: 1.00±0.03 and 0.98±0.03, respectively). This confirms the presence of large defects in the membrane, and explains the lack of retention of cytoplasmic signaling proteins. Reduced penetration of dextrans is observed starting from molecular weight 250 kDa (intensity ratio: 0.84±0.05). Dextrans are known to form rod-like structures in aqueous solutions, and hydrodynamic radii for the 20, 73, 250, 500, and 2000 kDa dextrans have been reported as 3.2, 6.5, 11.5, 15.9, and 26.9 nm, respectively. Taking into account that the hydrodynamic radius for an IgG antibody (150 kDa) is 5.4 nm, and thus falls in between the hydrodynamic radii of 20 kDa and 70 kDa dextran, we predict that antibodies which are added externally will penetrate the vesicles. This makes it possible to detect specific phosphotyrosines on the kinase domain of an RTK in the vesicle lumen as illustrated in FIG. 9. The antibodies are recruited to the vesicle membrane upon tyrosine phosphorylation, where their fluorescence intensities are measured.

Vesicles were produced from cells transfected with EGFR. Experiments were set up with 100 nM EGF in the presence of an ATP cocktail containing Mg²⁺ and a phosphatase inhibitor (see Methods). A FITC-labeled anti-pY 4G10 antibody, which recognizes any phos-phorylated tyrosine residue on EGFR in response to EGF stimulation, was used for detection. An IgG-FITC isotype control antibody was used as a control.

FIG. 1C-FIG. 1D show imaging of fluorescence of FITC-labeled anti-pY 4G10 antibodies and igG-FITC isotype control antibodies (FIG. 1C) and the corresponding graph of fluorescence over time (min) (FIG>1D). FIG. 1C shows EGFR phosphorylation in vesicles. First column: receptor channel, middle column: antibody channel, right column: overlay. (i) Vesicles derived from CHO cells with EGFR-mTurq incorporated into the vesicle membrane were incubated with 10 μg/mL IgG-FITC isotype control antibody. (ii) Vesicles derived from CHO cells with EGFR-mTurq incorporated into the vesicle membrane were incubated with 100 nM EGF, ATP/salt cocktail, and FITC anti-pY 4G10 antibody. The antibody fluorescence can be seen on the membrane. Representative images from 3 independent experiments with the 4G10 antibody. While no significant binding to the vesicle membrane was detected for the control antibody, a clear increase in membrane fluorescence was observed for the anti-pY antibody upon EGF addition (FIG. 1C). In both cases the solution fluorescence intensity of the antibody fluorophores is equal inside and outside the vesicles, showing that antibodies are able to freely diffuse across the vesicle membrane.

FIG. 1D shows phosphorylation signal on the vesicle membrane over time. The fluorescence of the antibody was measured on the membrane of a single vesicle over time in response to 10 nM EGF with added ATP cocktail (red line). The black line shows a control experiment in the absence of EGF and ATP. The maximum antibody binding and therefore receptor phos-phorylation is achieved after about 20 min, with a time course which is likely affected by ligand and antibody diffusion (FIG. 1D). Importantly for this work, a plateau in the fluorescence was observed, demonstrating that the phosphorylation comes to equilibrium, consistent with the expectation that no feedback loops are present. As a control, no increase in fluorescence was observed in the absence of ligand and ATP kinase cocktail.

### Example 2: Quantification of Novel Intrinsic Ligand Bias and Quantification of Novel Mutation-Induced Signaling Bias

### EGFR Phosphorylation in CHO Vesicles

To measure the phosphorylation of EGFR in the vesicle membranes, the vesicles were transferred to an 8-well glass bottom chamber slide (ibidi, #80827). The final concentrations of the ATP cocktail ingredients were 1 nM ATP, 10 mM MgCl2, and 0.1 mM Na₃ VO₄, a phosphatase inhibitor. The ligands used in this study were EGF (8916sf, Cell Signaling), TGFα (239A100, R&D Systems), Epiregulin (1195EP025 CF, R&D Systems), and EGF-tetramethylrhodamine (E3481, Thermofisher). For the transducer function measurements, we used commercially available EGF ligand from mouse that is labeled with rhodamine at its N-terminus (rho-mEGF, Thermofisher, E3481).

For detection of any phosphorylated Y residues, we used 67 nM of FITC-labeled anti-pY 4G10 antibody (05-321, Sigma Aldrich). For detection of Y1068 phosphorylation, 233 nM of AlexaF488-labeled anti-pY1068 EGFR antibody (IC3570G100, R&D Systems) was added. Y1173 phosphorylation was detected using 50 nM AlexaF488-labeled anti-pY1173 EGFR antibody (NBP1-44893AF488, Novus Biologicals).

Concentrations of the antibodies were chosen such that (i) the fluorescence intensities can be detected and measured on the plasma membrane (this depends on the labeling of the antibodies), (ii) the antibody amount exceeds the total amount of EGFR in the sample and (iii) the antibody concentration exceeds the anti-pY antibody dissociation constants (low nM). To determine the total EGFR concentration in a chamber slide well, 100 μl EGFR-mTurq vesicles were transferred to 96-well plates and full fluorescence emission spectra were collected with a H4 Synergy Hybrid Microplate Reader (BioTek Instruments, Winooski, VT). The samples were excited at 430 nm with a 9 nm bandwidth and the emitted fluorescence was collected from 450 to 620 nm with a 9 nm bandwidth with 5 nm steps. The emission spectra were corrected by subtracting the emission spectra of a vesicle sample derived from untransfected CHO cells. The maximum intensity of the corrected emission spectra at 475 nm was used to calculate the total EGFR concentration in a well upon calibration with purified solutions of mTurq of known concentration.

To monitor the reaction kinetics of EGFR phosphorylation, image acquisition was started right after the addition of the ligand/ATP cocktail to the vesicles. For dose response measurements, the phosphorylation reaction was allowed to reach equilibrium for 1 h prior to image acquisition. Image acquisition was automated by selecting predefined regions and focus points in the LAS X Navigator software (Leica Biosystems, Wetzlar, Germany). The reaction was monitored for up to 5 h. About 5000 images per experiment were acquired.

All images were acquired with a TCS SP8 confocal microscope (Leica Biosystems, Wetzlar, Germany) equipped with a motorized stage and a HyD hybrid detector in photon counting mode. Two scans per vesicle were taken, an ‘mTurq’-scan (A=448 nm, emission window: 460-510 nm), where m Turq bound to EGFR is excited, and an ‘AlexaF488’-scan (A=488 nm, emission window: 500-540 nm), where AlexaF488 bound to the anti phospho antibody is excited. The images (512×512 pixels) were acquired at 1% laser power with a 50 Hz scanning speed. Under these conditions, measured bleed-through coefficients were: mTurq in AlexaF488 channel <0.8%: AlexaF488 in mTurq channel <2.5%. These were considered negligible.

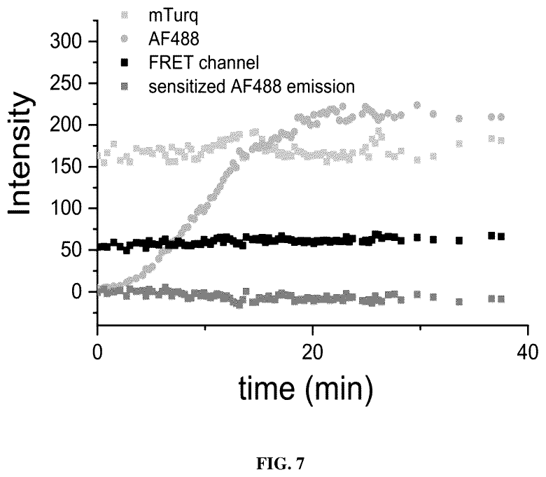

In experiments with unlabeled ligands, an mTurq/AlexaF488 FRET scan was performed: excitation: 448, emission: 500-540, to monitor if FRET occurs between mTurq and AlexaF488. Bleed through of mTurq into the FRET channel was 33%, and of AlexaF488 (due to direct excitation) was 7%. These values were used to determine the sensitized AlexaF488 fluorescence due to FRET between mTurq at the C-terminus of EGFR, and AlexaF488 on the antibody. FRET was negligible (FIG. 7), and thus no correction for FRET was required.

In experiments with labeled ligand, the third scan was: excitation: 552, emission: 565-625, 3% laser power. The bleed-throughs of rho-damine in the mTurq and AlexaF488 channel were both <1.5% and were considered negligible.

### Ligand Bias Analysis

Dose response curves were fitted with the Hill equation with a slope of 1 (Equation 41), as prescribed for calculations of the bias coefficient β_{lig}. The best fit EC₅₀ and Eₜₒₚ were used to calculate β_{lig} according to:

$${β}_{lig} = log ( {( \frac{{E}_{top , A} {EC}_{50 , B}}{{EC}_{50 , A} {E}_{top , B}} )}_{lig} {( \frac{{E}_{top , B} {EC}_{50 , A}}{{EC}_{50 , B} {E}_{top , A}} )}_{ref} ) \qquad ( 1 )$$

where response A is Y1068 phosphorylation and response B is Y1173 phosphorylation. The mutation-induced signaling bias coefficient was calculated as:

$${β}_{mut} = log ( {( \frac{{E}_{top , A} {EC}_{50 , B}}{E {C}_{50 , A} {E}_{top , B}} )}_{L 834 R} {( \frac{{E}_{top , B} {EC}_{50 , A}}{{EC}_{50 , B} {E}_{top , A}} )}_{WT} ) \qquad ( 2 )$$

To test for ligand bias significance, a one-way ANOVA followed by Tukey's multiple comparisons test was performed using Graph-Pad Prism version 9.2.0 (GraphPad Software, San Diego, California USA).

The standard errors for bias coefficients and β′ values used in the statistical tests were derived from Monte-Carlo error estimations. For each parameter, 10⁶ normally distributed numbers were randomly generated using the mean and standard error of the parameter. The standard error of the distribution of the calculated bias coefficients was used for the statistical analysis.

### Quantification of Intrinsic Ligand Bias in EGFR Signal Propagation Across the Plasma Membrane

We investigated if there is preference for the phosphorylation of one of two tyrosines when EGFR is activated by three EGFR ligands: EGF, TGFα, and epiregulin. The two tyrosines that were probed, Y1068 and Y1173, are in the long unstructured tail of EGFR and have profound importance for signaling. Phosphorylation of Y1068 leads to the recruitment of Grb2 and Gab1 and the activation of AKT and STAT3/5 signaling pathways. On the other hand, Y1173 phosphorylation leads to the recruitment of Shc and the activation of the MAPK/ERK signaling cascade (although there is cross-talk between the different pathways which is cell-specific). The differential phosphorylation of these two tyrosines is believed to lead to different functional outcomes, and their differential phosphorylation in cells has already been used as an indicator of functional selectivity in EGFR signaling.

Experiments were performed with EGFR-mTurquoise (EGFR-mTurq), in which the fluorescent protein mTurq was attached to the C-terminus of EGFR via a 15 aa linker. This attachment does not impact the activation of EGFR. The cells were vesiculated and thousands of individual vesicles were imaged. To detect EGFR phosphorylation, we used either anti-pY1068 or anti-pY1173 EGFR antibody, labeled with AlexaF488. The molar concentration of the antibodies always exceeded at least 5 times the total molar concentration of EGFR (−10 nM) and the pY-antibody dissociation constant (low nM). To start the reaction, we added ligands together with ATP kinase cocktail (1 mM ATP, 0.5 mM DTT, 10 mM MgCl₂, 0.1 mM Na₃ VO₄ (a phosphatase inhibitor)). The antibody was recruited to the vesicle membrane and the recruitment was quantified through the increase in membrane fluorescence. The imaging was performed at least 1 h after the beginning of the reaction, based on kinetic traces of single vesicles which show complete equilibration after-20 min. Each vesicle was imaged using an automated microscope stage in two scans: one exciting mTurq at the C-terminus of EGFR, to assess EGFR concentration in each vesicle, and one exciting the fluorophore on the anti-pY antibody, to assess its concentration on the membrane in each vesicle. The degree of phosphorylation, per EGFR molecule, is thus proportional to the fluorescence ratio in the antibody channel and the EGFR channel.

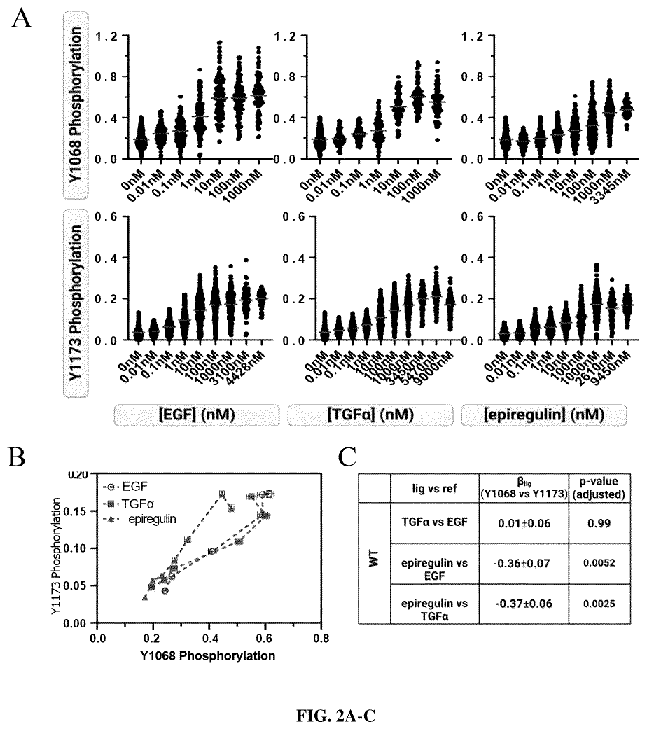

Complete dose-response curves for WT EGFR Y1068 and Y1173 phosphorylation per EGFR molecule, in response to EGF, TGFα, and epiregulin, were collected. FIG. 2A shows raw dose response curves for Y1068 and Y1173 phosphorylation per EGFR molecule, for the ligands EGF, TGFα, and epiregulin. Each point represents the ratio of either anti-pY1068 or anti-pY1173 fluorescence and EGFR-mTurq fluorescence for one individual vesicle. Each curve contains −1000 to −3000 data points (single vesicles). A total of 11,570 vesicles were imaged, while the concentration of different ligands was varied from zero to saturating concentrations. For each individual vesicle, on the y axis we report the ratio of: (i) the fluorescence of AlexaF488, linked to the anti-pY antibody and (ii) the fluorescence of mTurq, linked to the receptor. Thus, on the axis the values are proportional to the degree of EGFR phosphorylation, and on the x axis is the ligand concentration (FIG. 2A). As all measurements utilized the same microscope setting and the same antibody batches, all Y1068 data are on the same scale and all Y1173 data are on the same scale.

To determine if either Y1068 or Y1173 is preferentially phosphorylated by a ligand in comparison to another ligand, or whether there is no preference, we created bias plots. FIG. 2B shows ligand bias plots depicting three comparisons for the three ligands (TGFα vs EGF (reference), epiregulin vs EGF (reference), and epiregulin vs TGFα (reference)) and the two tyrosine phosphorylation responses (Y1173 phosphorylation and y1068 phosphorylation). Shown are means (symbols) and standard errors (often smaller than symbols). The epiregulin points diverge from the EGF and TGFα points. In total, data are from 11,570 single vesicles over 25 independent experiments. In the bias plots one response (pY1173) is plotted against the second response (pY1068) at the same ligand concentrations. These bias plots report directly on the relative effectiveness of the ligands to produce the two responses, without the need for assumptions or mathematical modeling. The bias plots in FIG. 2B are different, indicating that there is bias. In particular, the epiregulin points diverge from the EGF and TGFα points, in the direction of Y1173 phosphorylation. Thus, the bias plot in FIG. 2B demonstrates that epiregulin induces preferential phosphorylation of Y1173 over Y1068 when compared to EGF and TGFα.

Signaling bias due to a specific ligand can be identified and quantified with respect to a reference ligand by also calculating bias coefficients, such as the widely used β_{lig} given in Eq. (1). This requires that we know the potencies, EC₅₀, and the efficacies (maximum effects), Eₜₒₚ, of the ligand and the reference ligand for the two responses, pY1068 and pY1173. The coefficient β_{lig} has a sign that indicates the preference of the ligand, as compared to the reference ligand, for a particular response (+, if the first response is preferred and −, if the second response is preferred), as well as a magnitude which reports on the degree of bias. The case of β_{lig}=0 indicates that the ligand is not biased when compared to the reference ligand.

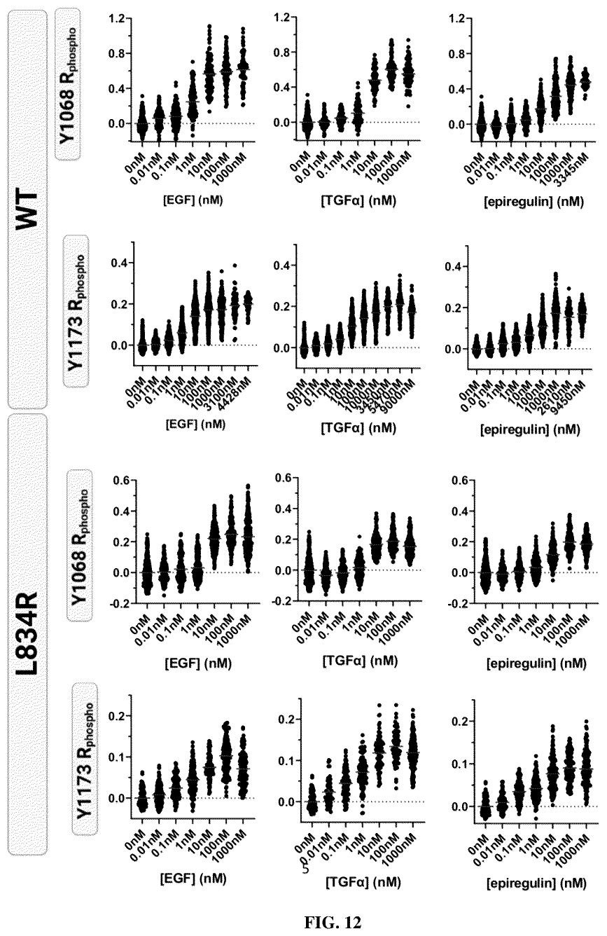

To calculate ECs and Eₜₒₚ for the three ligands, we first note that the phosphorylation at zero ligand in FIG. 2A is not zero. This is consistent with prior work, showing that EGFR can be phosphorylated in the absence of ligand, but cannot trigger downstream signaling, likely because the structure of the unliganded EGFR dimer is different from the structure of the active ligand-bound EGFR dimers. To characterize the response to ligand, we corrected the measured dose responses for the contribution of the unliganded dimers as described in Example 5 (FIG. 12). The best-fit potencies (EC₅₀) and the efficacies (Eₜₒₚ), calculated using a Hill slope of 1, are given in Table 1:

TABLE 1
EC₅₀ and Eₜₒₚ for the EGF, TGFα and epireglin responses.
Best-fit values ± SEM are shown.

| | | ligand | EC₅₀ (nM) | Eₜₒₚ |
|---|---|---|---|---|
| WT | Y1068 | EGF | 1.4 ± 0.1 | 0.61 ± 0.01 |
| | | TGFα | 3.4 ± 0.3 | 0.59 ± 0.01 |
| | | epiregulin | 19.5 ± 2.0 | 0.43 ± 0.01 |
| | Y1173 | EGF | 2.3 ± 0.1 | 0.19 ± 0.01 |
| | | TGFα | 5.2 ± 0.3 | 0.17 ± 0.01 |
| | | epiregulin | 16.2 ± 1.2 | 0.16 ± 0.01 |
| L858R | Y1068 | EGF | 3.1 ± 0.3 | 0.25 ± 0.01 |
| | | TGFα\| | 3.2 ± 0.4 | 0.17 ± 0.01 |
| | | epiregulin | 5.8 ± 0.6 | 0.20 ± 0.01 |
| | Y1173 | EGR | 0.7 ± 0.1 | 0.08 ± 0.01 |
| | | TGFα | 0.3 ± 0.1 | 0.12 ± 0.01 |
| | | epiregulin | 0.7 ± 0.1 | 0.09 ± 0.01 |

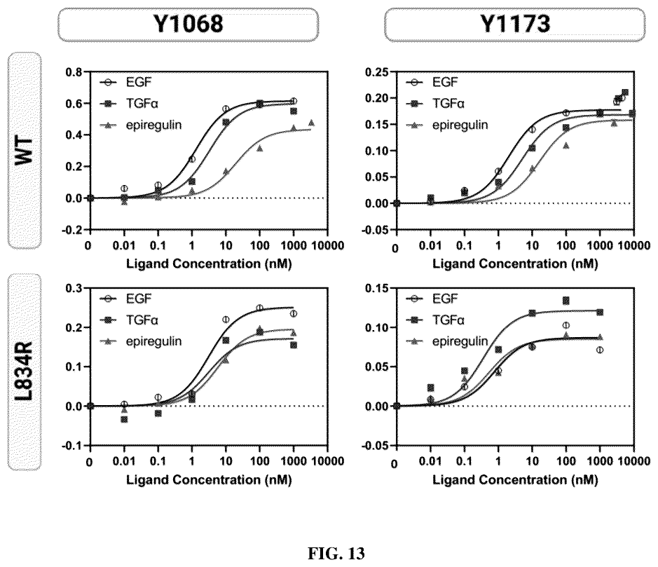

The corrected averaged dose response curves are shown in FIG. 13.

TABLE 2
EC₅₀ and Eₜₒₚ for the rho-mEGF response.
Best-fit values ± SEM are shown.

| | EC₅₀ (nM) | Eₜₒₚ |
|---|---|---|
| Y1068 | 5.5 ± 0.3 | 0.67 ± 0.01 |
| Y1173 | 6.8 ± 0.4 | 0.21 ± 0.01 |

FIG. 2C shows a table of three ligand bias coefficients for the three comparisons (TGFα vs EGF (reference), epiregulin vs EGF (reference), and epiregulin vs TGFα (reference)) using equation 1, and standard errors. Epiregulin is biased toward Y1173 phosphorylation as compared to EGF and TGFα. Ordinary one-way ANOVA, followed by Tukey's test, was used to determine statistical significance. The p values are adjusted for multiple comparisons. This demonstrated the presence of epiregulin-induced bias towards Y1173 phosphorylation when compared to EGF (p=0.005) and TGFα (p=0.003). This analysis is entirely consistent with the bias plots, which are created by plotting the experimentally measured data without any corrections or assumptions.

### The Common NSCLC L834R (L858R) Driver Mutation in EGFR Induces Intrinsic Bias in Signal Propagation Across the Plasma Membrane

NSCLC represents over 85% of all lung cancers and is associated with high mortality. The 5-year survival for all stages of progression is <17%. This cancer is due to EGFR mutations in-10-15% of Caucasian patients and in up to 50% of Asian patients. Of the single amino acid mutations, the L834R mutation is the most common one, accounting for about 40-45% of the cases where EGFR is mutated. (This mutation is often referred to as the “L858R mutation” when the EGFR signal peptide is counted).

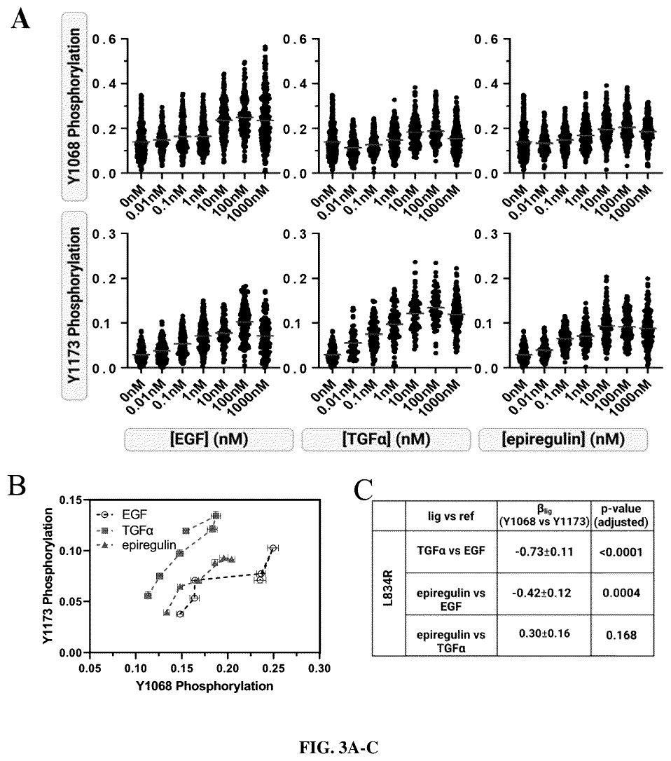

We acquired dose response curves for L834R EGFR in response to EGF, TGFα, and epiregulin. (FIG. 3A shows single vesicle dose response curves for Y1068 and Y1173 phosphorylation per EGFR molecule, for EGF, TGFα, and epiregulin. Each point represents the ratio of either anti-pY1068 or anti-pY1173 fluorescence and EGFR-mTurq fluorescence for one individual vesicle. Each curve contains −1000 to −1800 data points.

A total of 8009 individual vesicles were imaged and analyzed in these experiments (FIG. 3A). (FIG. 3A shows single vesicle dose response curves for Y1068 and Y1173 phosphorylation per EGFR molecule, for EGF, TGFα, and epiregulin. Each point represents the ratio of either anti-pY1068 or anti-pY1173 fluorescence and EGFR-mTurq fluorescence for one individual vesicle. Each curve contains −1000 to −1800 data points.)

We then constructed ligand bias plots (FIG. 3B), (FIG. 3B shows bias plots. Shown are means (symbols) and standard errors (often smaller than symbols). In total, data are from 8009 vesicles in 23 independent experiments.)

In the case of L834R EGFR, both TGFα and epiregulin are biased toward Y1173 phosphorylation over Y1068 phosphorylation, as compared to EGF. Thus, the relative bias of the three EGFR ligands is altered due to the L834R mutation. These conclusion from the bias plots are supported by the calculations of bias coefficients (FIG. 3C) and their statistical analysis. (FIG. 3C shows bias coefficients and their standard errors. Ordinary one-way ANOVA, followed by Tukey's test, was used to determine statistical significance. The p values are adjusted for multiple comparisons.) The corrected averaged dose response curves are shown in FIG. 13.

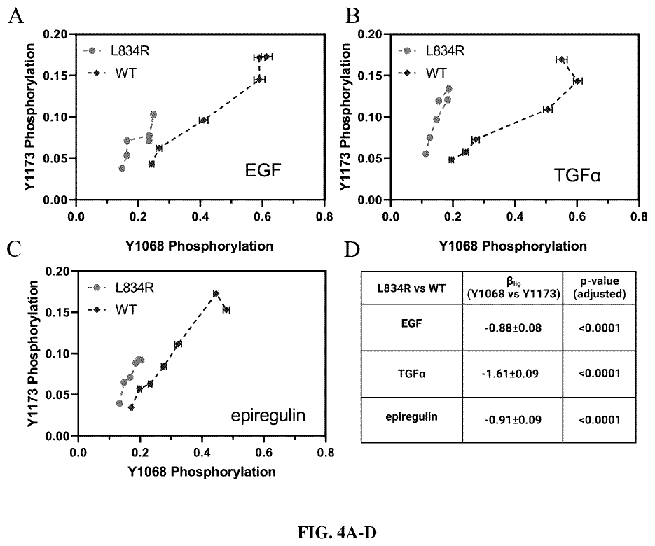

To directly answer the question if the mutation causes bias in EGFR signaling, we created bias plots while directly comparing the wild-type and the mutant (FIG. 4A, FIG. 4B, and FIG. 4C). FIG. 4A-FIG. 4C show L834R mutation-induced signaling bias plots in the presence of EGF, TGFα, and epiregulin. Shown are means (symbols) and standard errors (often smaller than symbols). In total, data are from 11,570 single vesicles over 25 independent experiments for WT and 8009 vesicles for L834R EGFR in 23 independent experiments.

These are mutation-induced signaling bias plots, distinctly different from the ligand bias plots in FIG. 2B and FIG. 3B, as they now directly compare the mutant and the wild-type. In FIG. 4A-C we see that the mutation induces significant preference for Y1173 phosphorylation over Y1068 phosphorylation, when compared to the wild-type, in the presence of the three ligands. We then calculated a different type of bias coefficient, the mutation-induced signaling bias coefficient βₘᵤₜ, to quantify the degree of bias introduced by the mutation in EGFR signaling in response to a specific ligand. The values were calculated using Eq. (2), where “response A” refers to Y1068 phosphorylation and “response B” refers to Y1173 phos-phorylation (FIG. 4D). FIG. 4D shows bias coefficients and their standard errors. Three pair-wise comparisons were performed using two-tailed t-tests. The p values were adjusted for multiple comparisons using the Holm-Sidak correction. The L834R mutation induces statistically significant preference for Y1173 phosphorylation over Y1068 phosphorylation, as compared to the wild-type, in the presence of all three ligands.

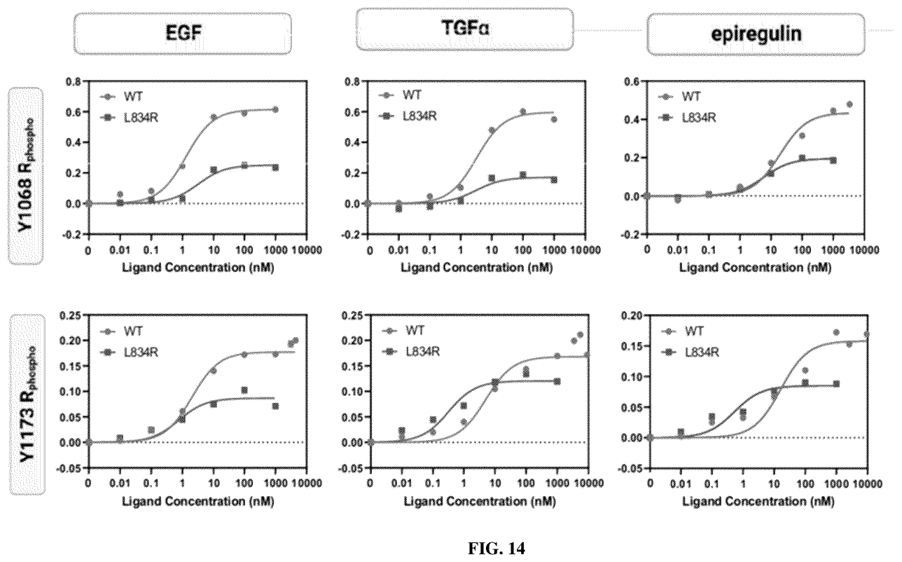

The effect is largest in the case of TGFα, but highly statistically significant for all ligands, based on t-tests. This is a direct demonstration that the L834R mutation induces signaling bias in EGFR phosphorylation in the plasma membrane for all studied ligands. The corrected averaged dose response curves for the wild-type and the mutant are compared in FIG. 14.

### Example 3: Quantification Receptor Tyrosine Kinase Phosphorylation Upon Ligand Stimulation and Determination of Transducer Function

### The Transducer Function

The transducer function that we measure for the first time informs whether an agonist can be improved further. Before, the maximal receptor response was unknown and therefore it was impossible to compare agonists on an absolute scale.

The transducer function relates a response to the stimulus that is causing it. Experimentally, signaling responses downstream of a receptor depend on the abundance of activated receptors through a hyperbolic dependence. The hyperbolic dependence was derived from first principles by Black and Leff, and is the basis for their Operational Model, which is valid for different types of receptors including RTKs. In this model, the ligand-bound receptors act as a stimulus that activates the response with an effective equilibrium dissociation constant denoted as Kᵣₑₛₚ′:

$$response = transducer function ( stimulus ) = \frac{stimulus {R}_{max}}{stimulus + {K}_{resp}^{′}} \qquad ( 3 )$$

In our experiments the “response” is the phosphorylation of a tyrosine in the intracellular domain of an RTK, Rₚₕₒₛₕₒ. We denote the maximum possible phosphorylation signal that can be achieved for this tyrosine in response to a full agonist as Rₘₐₓ.

The “stimulus” is the concentration of the ligand-bound receptors, [RL]. Therefore:

$${R}_{phospho} = \frac{[ RL ] {R}_{max}}{[ RL ] + {K}_{{resp}^{′}}} \qquad ( 4 )$$

If we divide both the numerator and denominator by the total receptor concentration, [Rt] and denote the fraction bound receptors, [RL]/[Rt], as f_{bound}, we obtain:

$${R}_{phospho} = \frac{{f}_{bound} {R}_{max}}{{f}_{bound} + {K}_{resp}} \qquad ( 5 )$$

$$where$$

$${K}_{resp} = \frac{{K}_{{resp}^{′}}}{[ Rt ]} \qquad ( 6 )$$

and the “stimulus” is now redefined as the fraction of ligand-bound receptors, f_{bound}. Kᵣₑₛₚ is the fraction of ligand-bound receptors that yields 50% of Rₘₐₓ. The value of Rₘₐₓ depends on the fluorescent properties of antibodies used for the detection and thus the phosphorylation response is fully described by the ratio of Rₚₕₒₛₚₕₒ/Rₘₐₓ:

$$\frac{{R}_{phospho}}{{R}_{max}} = \frac{{f}_{bound}}{{f}_{bound} + {K}_{resp}} \qquad ( 7 )$$

The ligand-bound fraction f_{bound} varies between 0 and 1. Setting f_{bound}=1, we define:

$$phosphorylation efficiency = \frac{{R}_{phospho} ( {f}_{bound} = 1 )}{{R}_{max}} = \frac{1}{1 + {K}_{resp}} \qquad ( 8 )$$

This efficiency describes the maximum phosphorylation per receptor that can be achieved in response to a specific ligand, when all receptors are ligand-bound. It can be determined if the transducer function, given by Eq. (4), is measured experimentally and Rₘₐₓ and Kᵣₑₛₚ are determined from a two-parameter fit. The smaller the value of Kᵣₑₛₚ, the more efficient the phosphorylation. Phosphorylation efficiency of→1 (Kᵣₑₛₚ→0) is indicative of a full agonist.

A measurement of the phosphorylation transducer function. The transducer function relates a response to the stimulus that is causing it. In our case, the response is the phosphorylation of a tyrosine in the intracellular domain of an RTK. The stimulus is the formation of the ligand-bound RTK dimers. We therefore sought to measure both ligand binding and phosphorylation simultaneously so we can plot one vs the other and obtain the transducer function.

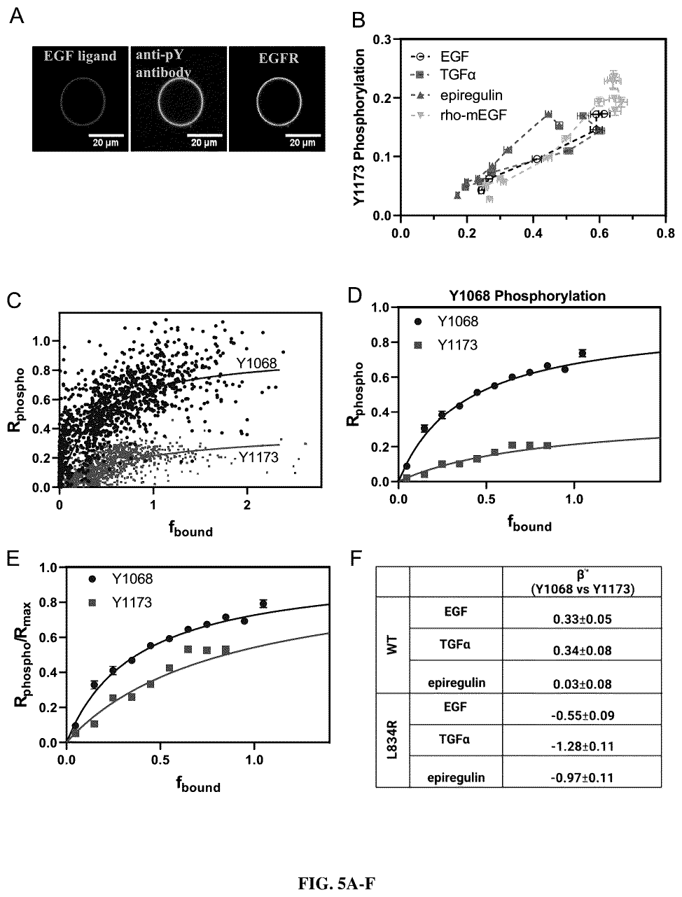

To demonstrate the feasibility of transducer function measurements, we used commercially available EGF ligand from mouse that is labeled with rhodamine at its N-terminus (rho-mEGF, Thermofisher, E3481), a ligand that binds human EGFR with 3 times lower affinity than human EGF. To determine the transducer function for rho-mEGF, individual vesicles were imaged in a confocal microscope in three scans to measure: (i) the fluorescence of rhodamine, linked to mEGF, on the membrane, to quantify the bound ligand in the plasma membrane in each vesicle Ex: 552 nm; Em: 565-625 nm, (ii) the fluorescence of AlexaF488, linked to the anti-phosphoY antibody, to quantify phosphorylated EGFR in the membrane Ex: 488 nm; Em: 500-540 nm (iii) the fluorescence of mTurq, linked to the receptor, to quantify EGFR in the plasma membrane in each vesicle Ex: 448 nm; Em: 460-510 nm. One vesicle, imaged in the three scans, in the presence of 5 nM EGF, is shown in FIG. 5A. (FIG. 5A shows one vesicle imaged in three channels at 5 nM rho-mEGF in the presence of ATP/kinase cocktail.) More than 3000 vesicles were imaged, while the ligand concentration was varied from zero to saturating concentrations.

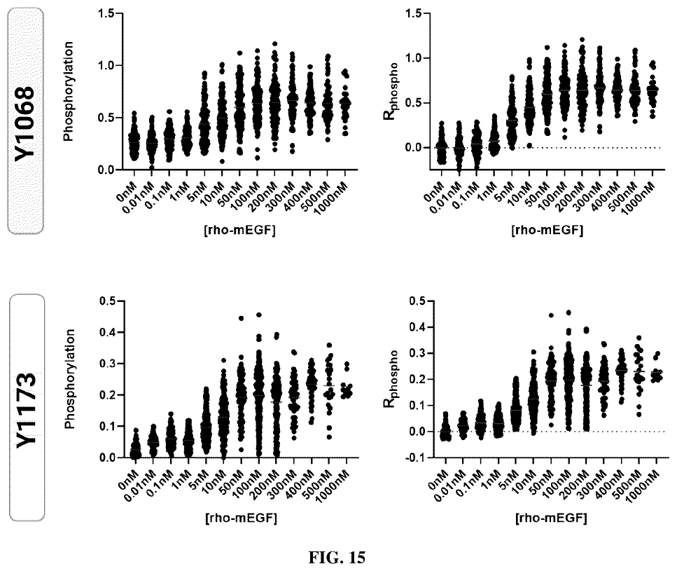

The mouse and human EGF differ in sequence and affinity to human EGFR (Example 7). To assess if the mouse rho-EGF induces biased EGFR signaling, as compared to the three human ligands, we used the acquired phosphorylation dose response curves shown in FIG. 15 to construct bias plots comparing rho-mEGF and the human ligands (FIG. 5B), and we calculated bias coefficients in Table 3:

TABLE 3
Calculated Rho-mEGF bias coefficients. Ordinary one-
way ANOVA was used for p-value calculation. Tukey test
was used to account for multiple comparisons. Reported
are p values adjusted for multiple comparisons.

| lig vs | β_{lig} | p-value |
|---|---|---|
| rho-mEGF | (Y1068 vs Y1173) | (adjusted) |
| EGF | 0.12 ± 0.05 | 0.48 |
| TGFα | 0.13 ± 0.06 | 0.29 |
| epiregulin | −0.23 ± 0.06 | 0.0001 |

By ANOVA, the two EGF ligands are not biased, despite the reported differences in affinity to EGFR. (FIG. 5B shows bias plots for rho-mEGFR and human EGF, TGFα, and epiregulin. EGF and rho-mEGF are not biased ligands. Shown are means and standard errors. In total, data are from 22,670 single vesicles over 34 independent experiments.)

In FIG. 5C, we plot the phosphorylation response (fluorescence in the antibody channel divided by the fluorescence in the EGFR channel) as a function of the stimulus (ligand-bound EGFR fraction, f_{bound},) for each individual vesicle that was imaged in the three channels. The x axis (fluorescence in the ligand channel divided by the fluorescence in the EGFR channel) is scaled such that the maximum average fraction of ligand-bound EGFR is set to 1 and the y axis is phos-phorylation corrected for constitutive (ligand-independent)phosphorylation. This plot represents the transducer function. The solid lines are the transducer function fits (Eq. (5) to all the single vesicle data. Data are from 2173 individual vesicles for Y1068 and 1987 individual vesicles for Y1173 over 9 independent experiments in which the concentration of rho-mEGF was varied.)

We fit the data in FIG. 5C using Eq. (5) to determine (i) Rₘₐₓ, the maximal possible signal that can be achieved in the experiment by a true full agonist (Kᵣₑₛₚ→0), which depends on the fluorescent properties of the antibodies and (ii) Kᵣₑₛₚ, the fraction of ligand-bound receptors that yields 50% of Rₘₐₓ. The smaller the value of Kᵣₑₛₚ, the more efficient the phosphorylation. The best-fit values for Y1068 phosphorylation are Kᵣₑₛₚ=0.40±0.03 and Rₘₐₓ=0.95±0.03. The best-fit values for Y1173 phosphorylation are Kᵣₑₛₚ=0.86±0.08 and Rₘₐₓ=0.39±0.02. In FIG. 5D, we show the single-vesicle data has been binned in an interval of 0.1 on the x axis and is shown along with the fits to all the single vesicle data. Only bins containing at least 50 vesicles are shown. Shown are standard errors; if not visible they are smaller than the symbols.

Kᵣₑₛₚ for Y1068 phosphorylation is the smaller of the two, indicating that Y1068 phosphorylation is more efficient than Y1173 phos-phorylation in response to EGF. Since the two Rₘₐₓ values differ because of the different fluorescent properties of the two antibodies, in FIG. 5E we plot the normalized transducer function, i.e., the dependence of Rₚₕₒₛₚₕₒ/Rₘₐₓ on the bound fraction, f_{bound}. (FIG. 5E shows the normalized transducer function, given by Eq. (7), and the calculated standard errors. Data are from 2173 individual vesicles for Y1068 and 1987 individual vesicles for Y1173 over 9 independent experiments.) The y value at f_{bound}=1 is the phosphorylation efficiency, calculated using Eq. (8) as 0.71±0.02 for Y1068 phosphorylation and 0.54±0.02 for Y1173 phosphorylation. Thus, rho-mEGF is a partial agonist for both responses. Human EGF, TGFα, and epiregulin are also partial agonists, as the Eₜₒₚ values for their responses do not exceed the ones for rho-mEGF (see Tables 1 and 2).

### Example 4: Absolute Bias Coefficients

### Calculation of Absolute Bias Coefficients

FIG. 5F shows absolute bias coefficients calculated using Eqs. (19)-(23)).

Bias coefficients calculated using Eqs. (1) and (2) are relative, i.e., β_{lig} is always calculated with respect to the reference ligand in the literature. However, the effective equilibrium constant Kᵣₑₛₚ can be used to calculate absolute bias coefficients, β/*_{lig} and β/*_{ref} (see Eqs. (22) and (23)). First, we calculate β/*_{rho-mEGF} using Eq. (20); β/*_{rho-mEGF}=0.33±0.5. Since this ligand is not biased in comparison to EGF, β/*_{EGF}=β/*_{rho-mEGF}. The absolute β/*_{EGF} directly reports on the preference of a ligand toward either Y1068 or Y1173 phosphorylation. The value of β/*_{EGF} is positive, indicating that Y1068 is preferentially phosphorylated in response to EGF, as compared to Y1173.

With the values of β/*_{lig} and β/*_{EGF} known, we calculate the absolute bias coefficients β/*_{TGFα} and β/*_{epiregulin}, for TGFα and epiregulin, using Eq. (22). Similarly, we calculate the absolute bias coefficients β/*ₘᵤₜ for the L834R mutant using Eq. (23). All absolute bias coefficients are shown in FIG. 5F. We see that Y1068 in WT EGFR is preferentially phosphorylated in response to EGF and TGFα, but there is no preference in response to epiregulin. The signaling of the mutant is always biased toward Y1173 phosphorylation.

The relation between Kᵣₑₛₚ and bias, and the definition of absolute bias coefficients Corrected dose-response curves are fitted using the Hill equation with n=1 (Equation 42). The Black and Leff operational model is consistent with Equation 42, but also provides a physical-chemical description of the activation process. According to the Black and Leff model, the concentration of the ligand-bound receptors [RL] in Eq. (4) depends on the concentrations of free receptor [R] and ligand [L], and on the effective ligand-receptor dissociation constant K_{L} according to the equation:

$$[ RL ] = \frac{[ R ] [ L ]}{{K}_{L}} \qquad ( 9 )$$

The total receptor concentration [Rt] is:

$$[ Rt ] = [ R ] + [ RL ] \qquad ( 10 )$$

Therefore:

$$[ RL ] = \frac{\frac{[ Rt ] [ L ]}{{K}_{L}}}{1 + \frac{[ L ]}{{K}_{L}}} \qquad ( 11 )$$

Substitution of Eq. (11) into Eq. (4) yields:

$$response = \frac{[ Rt ] [ L ] {R}_{max}}{[ Rt ] [ L ] + {K}_{{resp}^{′}} ( {K}_{L} + [ L ] )} = \frac{[ Rt ] [ L ] {R}_{max}}{[ L ] ( [ Rt ] + {K}_{{resp}^{′}} ) + ( {K}_{L} {K}_{{resp}^{′}} )} \qquad ( 12 )$$

Dividing both the numerator and the denominator by Kᵣₑₛₚ′, we obtain:

$$response = \frac{( \frac{[ Rt ]}{{K}_{{resp}^{′}}} ) [ L ] {R}_{max}}{( \frac{[ Rt ]}{{K}_{{resp}^{′}}} + 1 ) [ L ] + ( {K}_{L} )} = \frac{τ [ L ] {R}_{max}}{( τ + 1 ) [ L ] + ( {K}_{L} )} \qquad ( 13 )$$

where r is the “transduce coefficient” defined as:

$$τ = \frac{[ Rt ]}{{K}_{{resp}^{′}}} \qquad ( 14 )$$

Equation (13) can also be written as:

$$response = \frac{τ / ( τ + 1 ) [ L ] {R}_{max}}{[ L ] + ( {K}_{L} ) / ( τ + 1 )} \qquad ( 15 )$$

Now we see that Eq. (15) is the same as Equation 42, where

$${E}_{top} = \frac{τ {R}_{max}}{( τ + 1 )} \qquad ( 16 )$$

$${EC}_{50} = \frac{{K}_{L}}{( τ + 1 )} \qquad ( 17 )$$

We use Eqs. (16) and (17) to arrive at an alternate expression of the bias coefficient

$$\begin{matrix}{β}_{lig} = log ( {( \frac{{E}_{top , A} {EC}_{50 , B}}{{EC}_{50 , A} {E}_{top , B}} )}_{lig} {( \frac{{E}_{top , B} {EC}_{50 , A}}{{EC}_{50 , B} {E}_{top , A}} )}_{ref} ) \\ = log ( {( \frac{{τ}_{A} {K}_{L , B}}{{K}_{L , A} {τ}_{B}} )}_{lig} {( \frac{{τ}_{B} {K}_{L , A}}{{K}_{L , B} {τ}_{A}} )}_{ref} )\end{matrix} \qquad ( 18 )$$

Assuming that the ligand binding coefficient KL does not depend on the binding of the anti-pY1068 and anti-pY1173 antibodies (K_{L,A}=K_{L,B}), we arrive at

$$\begin{matrix}{β}_{lig} = log ( {( \frac{{τ}_{A}}{{τ}_{B}} )}_{lig} {( \frac{{τ}_{B}}{{τ}_{A}} )}_{ref} ) = log ( {( \frac{\frac{\text{?}}{\text{?}}}{\frac{\text{?}}{\text{?}}} )}_{lig} {( \frac{\frac{\text{?}}{\text{?}}}{\frac{\text{?}}{\text{?}}} )}_{ref} ) \\ = log ( {( \frac{{K}_{resp , B}}{{K}_{resp , A}} )}_{lig} {( \frac{{K}_{resp , A}}{{K}_{resp , B}} )}_{ref} ) \\ = log {( \frac{{K}_{resp , B}}{{K}_{resp , A}} )}_{lig} - log {( \frac{{K}_{resp , A}}{{K}_{resp , B}} )}_{ref} = {β}_{lig}^{′ *} - {β}_{ref}^{′ *}\end{matrix} \qquad ( 19 )$$

$$\text{?} \text{indicates text missing or illegible when filed}$$

Where we have used Eq. (6) and β/*_{lig} and β/*_{ref} are defined as

$$\begin{matrix}{β}_{lig}^{′ *} = log {( \frac{{E}_{top , A} {EC}_{50 , B}}{{EC}_{50 , A} {E}_{top , B}} )}_{lig} = log ( {( \frac{{K}_{resp , B}}{{K}_{resp , A}} )}_{lig} ) \\ = - log ( {( \frac{{K}_{resp , A}}{{K}_{resp , B}} )}_{lig} )\end{matrix} \qquad ( 20 )$$

$$\begin{matrix}{β}_{ref}^{′ *} = log {( \frac{{E}_{top , A} {EC}_{50 , B}}{{EC}_{50 , A} {E}_{top , B}} )}_{ref} = log ( {( \frac{{K}_{resp , B}}{{K}_{resp , A}} )}_{ref} ) \\ = - log ( {( \frac{{K}_{resp , A}}{{K}_{resp , B}} )}_{ref} )\end{matrix} \qquad ( 21 )$$

This definition of β/*_{lig} and β/*_{ref} does not include measurement bias and these coefficients report on the preference for phosphoryla-tion in absolute terms. If K_{resp′,B}>K_{resp′,A}, then the value of β/* is positive and response A is preferred. If K_{resp′,A}>K_{resp′,B}, then the value of β/* is negative and response B is preferred.

Once β/*_{ref} is known, we can calculate β/*_{lig} as:

$${β}_{lig}^{\text{/*}} = {β}_{lig} - {β}_{ref}^{\text{/*}} \qquad ( 22 )$$

By analogy, we can calculate the absolute bias coefficient β/*ₘᵤₜ as:

$${β}_{mut}^{\text{/*}} = {β}_{mut} - {β}_{ref}^{\text{/*}} \qquad ( 23 )$$

Although the subject matter has been disclosed herein in details with reference to various embodiments and features, it will be appreciated by a person with ordinary skill in the art that the embodiments and features described hereinabove are not intended to limit the presently disclosed subject matter, and that other variations, modifications and other embodiments will suggest themselves to those of ordinary skill in the art, based on the disclosure herein. The presently disclosed subject matter therefore is to be broadly construed, as encompassing all such variations, modifications and alternative embodiments within the spirit and scope of the claims hereafter set forth.

### Example 6: Identification of Vesicles Using Machine Learning, and Quantification of Their Fluorescence Intensities

In this work, we had to analyze confocal images of thousands of vesicles. To increase the through-put of data processing, we developed an approach that uses artificial intelligence. The trained neural network is able to separate “vesicles” from “bad vesicles” and “cell debris”. A “vesicle” is defined as a spherical vesicle, completely separated from its parent cell (FIG. 6A). Vesicles that are out of focus, cut off by image edges, non-circular, or attached to cells were classified as “bad vesicles”. Remnants of the cells after vesiculation were classified as “cell debris”.

For the neural network training, we used the imageLabeler app in Matlab to annotate rectangular regions in confocal micrographs for the classes (i) vesicle, (ii) bad vesicle, and (iii) cell debris. The resulting dataset of 1402 labeled objects was randomly shuffled and split into 70% training data and 30% test data. To build a neural network that identifies good vesicles, the FasterRCNN object detection network based on ResNet-18 was trained with the training data in Matlab. ResNet-18's res4b_relu layer was chosen as the feature extraction layer. The learning rate was set to 0.001 and images were augmented during the training process by random horizontal reflection. 15 predefined anchor boxes, calculated using Matlab's “estimateAnchorBoxes”, were used. Bounding box overlap ratios were set to 0.6-1 for positive training samples and to 0-0.3 for negative training samples to ensure a tight overlap with ground truth. The network was trained for a total of 10 epochs.

To train the network, we analyzed images of ˜1400 vesicles with FGFR3-eYFP, an RTK that has been used in prior work for method development (1, 2). FIG. 6A shows the result of running the neural network on an image of FGFR3-eYFP vesicles. The neural network was able to distinguish between good and bad vesicles over broad intensity ranges.

Once selected by the neural network, the vesicle images were processed with a Matlab program (3, 4), which finds the center of the vesicle and performs a check to ensure that only spherical, defect-free vesicles are included in the analysis. Once the center is found, the intensity profile as a function from the center is plotted and the membrane intensity is calculated (FIG. 6B). To accomplish this, the signal that originates from the membrane is fitted to a Gaussian function, and the background fluorescence to an error function (FIG. 6B).

We found that the vesicle analysis program could not be efficiently applied for images acquired with an automated stage, without the pre-selection performed by the neural network. The vesicle analysis program recognized vesicles based on a threshold that is referenced to the object of highest intensity (5). Many images from the automated imaging sessions contained multiple vesicles of different intensities, and the brighter vesicles precluded the identification of vesicle of weaker intensities.

After the neural network recognition, each sub-image of a good vesicle, such as the ones shown in FIG. 6A, was inputted into the vesicle analysis program. In this case, there was only a single vesicle per sub-image. Thus, each vesicle selected by the neural network was evaluated separately with a background threshold determined by its own intensity. As a result of this evaluation, each vesicle from the neural network was either rejected or analyzed further as shown in FIG. 6B.

To validate the neural network, we manually selected and cropped images from an automated imaging session and we ran the vesicle analysis program. In parallel, we used the neural network on the same data set to pre-select the images. After analysis, the receptor concentration was calculated for each vesicle (3) and the results are shown in FIG. 6C. The histograms of data in FIG. 6C show that the neural network performs similarly to a trained researcher who manually selects and crops images of good vesicles.

In the case of automatic imaging, many images did not include any good vesicles. These were rejected by the neural network and were never inputted into the vesicle analysis program, significantly speeding up the analysis. Thus, the addition of the neural network made the automatic imaging and analysis highly efficient.

The neural network approach was used as a first step in the quantification of both EGFR phosphorylation in FIG. 2A and FIG. 3A and of ligand binding in FIG. 5. It was also used to quantify the degree of penetration by dextrans into the vesicles in FIG. 1B. The dextran experiments demonstrated the presence of defects of vesicles, which ensured that the phosphorylated tyrosines are accessible to externally added antibodies.

The vesicle analysis code has been deposited under accession code: gitlab.com/hristovagroup/vesicle-analysis and is incorporated herein by reference. The code to correct for unliganded EGFR dimers has been deposited under accession code: gitlab.com/hristovagroup/unliganded-dimer-correction/-/tree/main and is incorporated herein by reference.

### Example 7: Correction of Dose Response Curves for Basal EGFR Phosphorylation

According to the canonical model of RTK activation, RTKs are monomeric in the absence of ligand and are crosslinked upon ligand binding, which brings their catalytic domains in close proximity (27). However, numerous studies have demonstrated that EGFR activation is much more complex. First, it has been reported that EGFR can form dimers even in the absence of ligand, and that these dimers are phosphorylated, although they are not able to initiate downstream signaling cascades (28). Second, there are reports that EGFR can form oligomers upon ligand binding (29, 30). However, other studies show that EGFR exists predominantly in dimeric form (31).

To collect dose-response curves, phosphorylation of EGFR on the vesicle membrane was measured, as illustrated in FIG. 9, over a broad range of ligand concentrations. These dose response curves, shown in FIG. 2A and FIG. 3A in the main text, capture the phosphorylation of unliganded dimers, single-liganded dimers, and double-liganded EGFR dimers. The unliganded dimers account for basal phosphorylation of EGFR at zero ligand. Since we are interested in the response of EGFR to ligand, we modeled the concentration of unliganded EGFR dimers, and we corrected the dose response curves for the basal phosphorylation. This was done in order to calculate bias coefficients and therefore provide quantitative support for the bias plots, which rely on visual inspection (26).

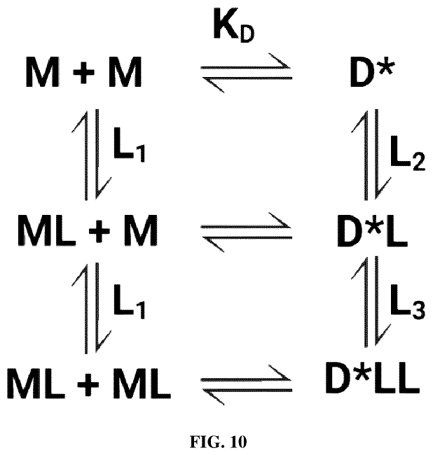

Despite open questions about the association state of EGFR, it has been shown that its function can be described in quantitative terms by the thermodynamic cycle in FIG. 10 which accounts for the coupling between ligand binding and EGFR dimerization (32). This cycle shows how increases in receptor and ligand concentration drive the formation of double-liganded EGFR dimers, and is used here to determine the unliganded dimer concentrations in each vesicle.

In our experiments, we know EGFR concentration in the membrane of each vesicle, [Rₜ]. We also know the total EGFR concentration in the imaging dish, [Rₜₒₜ], and the total ligand concentration in the imaging dish [Lₜₒₜ]. The dimerization constants K_{D} for EGFR and the L834R mutant have been measured as K_{D}=6.4×10−3 μm2 and 3.7×10−2 μm2, respectively, in the plasma membrane-derived vesicles (28). Noteworthy, the dimerization constant has also been measured in cells, and is the same (32). The EGF binding constants for the monomer, L1, the unliganded dimer, L2, and the liganded dimer, L3, have been measured as L1=4.6×10⁹M⁻¹, L2=5.3×10⁹M⁻¹, and L3=3.4×10⁸M⁻¹ (32). While both EGF and TGF are known as high-affinity ligands for EGFR, with similar association constants, epiregulin is known as a low-affinity ligand, and its association constant has been reported to be 46 times lower than that of EGFR (21, 33). The mouse EGF-human EGFR association constant is 3 times lower than the human EGF association constant (34).

Based on the cycle in FIG. 10, the concentrations of unliganded monomers, M, unliganded dimers, D, liganded monomers, ML, single-liganded dimers, DL, and double-liganded dimers, DLL, can be written as a function of these known equilibrium constants, the monomer concentration [M], and the free ligand concentration [L_{free}] according to:

$$[ D ] = {K}_{D} * {[ M ]}^{2} \qquad ( Eq . 24 ‐ 27 )$$

$$[ ML ] = {L}_{1} * [ {L}_{free} ] * [ M ]$$

$$[ DL ] = {L}_{2} * [ {L}_{free} ] * {K}_{D} * {[ M ]}^{2}$$

$$[ DLL ] = {L}_{2} * {L}_{3} * [ {L}_{free} ] * {K}_{D} * {[ M ]}^{2}$$

The total receptor concentration [Rₜ] can be written as:

$$[ {R}_{t} ] = [ M ] + [ ML ] + 2 [ D ] + 2 [ DL ] + 2 [ DLL ] = [ M ] + {L}_{1} * [ {L}_{free} ] * [ M ] + 2 {K}_{D} * {[ M ]}^{2} + 2 {L}_{2} * [ {L}_{free} ] * {K}_{D} * {[ M ]}^{2} + 2 {L}_{2} + {L}_{3} + {[ {L}_{free} ]}^{2} + {K}_{D} * {[ M ]}^{2} \qquad ( Eq . 28 )$$

which can be rewritten as:

$$[ {R}_{t} ] = ( 1 + {L}_{1} * [ {L}_{free} ] ) * [ M ] + ( 2 {K}_{D} + 2 {L}_{2} * {K}_{D} * [ {L}_{free} ] + 2 {L}_{2} * {L}_{3} * {K}_{D} * {[ {L}_{free} ]}^{2} ) * {[ M ]}^{2} \qquad ( Eq . 29 )$$

Thus, to be able to determine all the unknowns, we need to determine the free ligand concentration in the dish [L_{free}] and the EGFR monomer concentration [M] in each vesicle. First, we solve for the free ligand concentration in the dish. The total number of ligand molecules in the dish Lₜₒₜ is given by:

$${L}_{tot} = [ {L}_{free} ] * volume + {RL}_{tot} \qquad ( Eq . 30 )$$

where RLₜₒₜ the number of ligand molecules bound to receptors in the dish. ‘volume’ is the volume of the vesicle solution in the dish.

RLₜₒₜ is the sum of ligand molecules bound to monomers MLₜₒₜ, single-liganded dimers DLₜₒₜ, and double-liganded dimers DLLₜₒₜ:

$${RL}_{tot} = {ML}_{tot} + {DL}_{tot} + 2 {DLL}_{tot} \qquad ( Eq . 31 )$$

which can be written in terms of concentrations according to Equation 9:

$${RL}_{tot} = {R}_{tot} * ( \frac{[ {ML}_{tot} ]}{[ {R}_{tot} ]} + \frac{[ {DL}_{tot} ]}{[ {R}_{tot} ]} + \frac{2 [ {DLL}_{tot} ]}{[ {R}_{tot} ]} ) \qquad ( Eq . 32 )$$

Next, we assume that the average concentrations in the imaging dish is the same as the concentrations in a vesicle with an average total receptor concentration [R_{avg}]. We can write:

$$[ {R}_{avg} ] = \frac{{{\sum }_{i = 1}^{n} [ M ]}_{i} + {2 [ D ]}_{i} + {[ ML ]}_{i} + {2 [ DL ]}_{i} + {2 [ DLL ]}_{i}}{n} \qquad ( Eq . 33 )$$

$$[ {ML}_{avg} ] = \frac{{{\sum }_{i = 1}^{n} [ ML ]}_{i}}{n} \qquad ( Eq . 34 )$$

$$[ {DL}_{avg} ] = \frac{{{\sum }_{i = 1}^{n} [ DL ]}_{i}}{n} \qquad ( Eq . 35 )$$

$$[ {DLL}_{avg} ] = \frac{{{\sum }_{i = 1}^{n} [ DLL ]}_{i}}{n} \qquad ( Eq . 36 )$$

where [x]ᵢ is the concentration in the i^{th} vesicle, and n the total number of vesicles. We can substitute (Equation 11), (Equation 12) and (Equation 13) into (Equation 9) and write:

$${RL}_{tot} = {R}_{tot} * ( \frac{[ {ML}_{avg} ]}{[ {R}_{avg} ]} + \frac{[ {DL}_{avg} ]}{[ {R}_{avg} ]} + \frac{2 [ {DLL}_{avg} ]}{[ {R}_{avg} ]} ) \qquad ( Eq . 37 )$$

Note that in (Equation 14) the units of concentration are receptors per unit area instead of receptors per volume, as the receptors are confined in the 2-dimensional plasma membrane of the vesicle. Substituting (Equation 1), (Equation 2), (Equation 3), and (Equation 4) into (Equation 14), we obtain:

$${RL}_{tot} = {R}_{tot} * ( \begin{matrix}\frac{{L}_{1} * [ {L}_{free} ] * [ {M}_{avg} ]}{[ {R}_{avg} ]} + \frac{2 {L}_{2} * [ {L}_{free} ] * {K}_{D} * {[ {M}_{avg} ]}^{2}}{[ {R}_{avg} ]} + \\ \frac{4 {L}_{2} * {L}_{3} * {[ {L}_{free} ]}^{2} * {K}_{D} * {[ {M}_{avg} ]}^{2}}{[ {R}_{avg} ]}\end{matrix} ) \qquad ( Eq . 38 )$$

Substitution of (Equation 15) into (Equation 7) and further simplification yields:

$${L}_{tot} = {R}_{tot} * ( \frac{4 {L}_{2} * {L}_{3} * {K}_{D} * {[ {M}_{avg} ]}^{2}}{[ {R}_{avg} ]} ) * {[ {L}_{free} ]}^{2} + ( {R}_{tot} * ( \frac{2 {L}_{2} * {K}_{D} * {[ {M}_{avg} ]}^{2}}{[ {R}_{avg} ]} + \frac{{L}_{1} * [ {M}_{avg} ]}{[ {R}_{avg} ]} ) + volume ) * [ {L}_{free} ] \qquad ( Eq . 39 )$$

(Equation 16) and (Equation 6) are two equations for the two unknowns, [L_{free}] and [M_{avg}]. We use (Equation 6), written for [M_{avg}], to solve for [M_{avg}] and we substitute it in (Equation 16). Then, we solve (Equation 16) for [L_{free}] and we use this value to calculate [M] in each vesicle from (Equation 6). With [M] and [L_{free}] known, we calculate all other relevant concentrations using equations (Equation 1) to (Equation 4), for each vesicle.

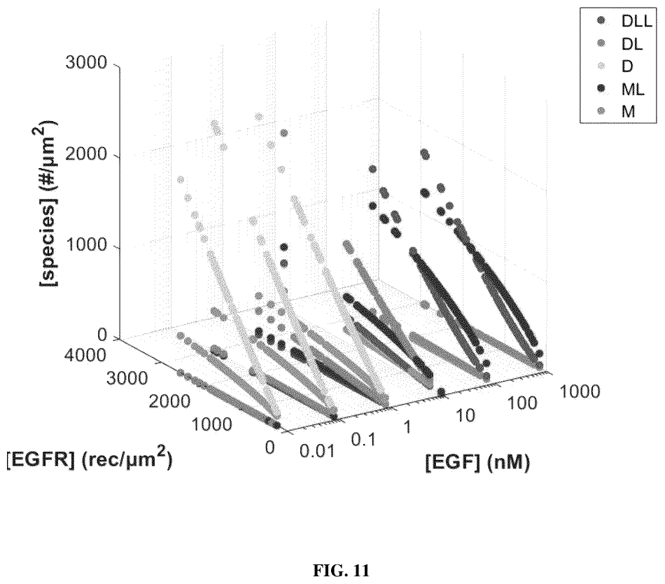

The calculated concentrations, for every vesicle in the case of WT EGFR in the presence of EGF are plotted in FIG. 11 as a function of the total ligand concentration and the total receptor concentration in the vesicles. At low ligand concentrations unliganded monomers and dimers dominate, while at high ligand concentrations mostly single-liganded monomers and double-liganded dimers are present. The presence of single-liganded dimers, DL, can only be observed at ligand concentrations around 10 nM EGF. The concentrations of unliganded dimers, [D], decreases with ligand concentration, as expected. Dividing each value of 2 [D] by the total receptor concentration in the vesicle, [Rt], gives us the fraction of receptors in the unliganded dimeric state, f_{D}, in the vesicle:

$${f}_{D} = \frac{2 [ D ]}{[ Rt ]} \qquad ( Eq . 40 )$$

When no ligand is added to the vesicles, the observed phosphorylation, pY1068 and pY1173, is exclusively due to unliganded dimers. To quantify EGFR activation in direct response to ligand binding, Rₚₕₒₛₚₕₒ, we fit the data in FIG. 2A and FIG. 3A in the main text to a Hill equation with n=1:

$$pY = {R}_{phosho} + k * {f}_{D} = \frac{[ {L}_{tot} ] * {E}_{top}}{{EC}_{50} + [ {L}_{tot} ]} + k * {f}_{D} \qquad ( Eq . 41 )$$

where fD is given by (Equation 17) and

$${R}_{phosho} = \frac{[ {L}_{tot} ] * {E}_{top}}{{EC}_{50} + [ {L}_{tot} ]} \qquad ( Eq . 42 )$$

Here, pY is the measured phosphorylation, given by the fluorescence intensity of the anti-pY antibody signal divided by the EGFR-mTurq fluorescence, as shown in FIG. 2A and FIG. 3A. k is the best-fit correction factor, which is determined in the fit, along with EC₅₀ and Eₜₒₚ. Note that equation (1) for bias coefficients is valid only in the case when Hill coefficient=1 (35), and thus we fix it to 1 in the fit.

The corrected dose response curves, Rₚₕₒₛₚₕₒ, are shown in FIG. 12. The averaged dose response curves and the fits to all the single vesicle data are shown in FIG. 13 and FIG. 14. The best-fit EC₅₀ and Eₜₒₚ values are shown in FIG. 2C and FIG. 3C.

The unliganded dimeric fraction, fD, in (Equation 18), depends on the ligand binding constants L1, L2, and L3 (FIG. 10). These constants have been measured for EGF (32). Here we use the same values for EGF and TGFD. Epiregulin is known as a low-affinity ligand, reported to bind 10 to 100 times weaker than EGF. By ITC, it has been shown to bind 46 times weaker (21). We use 46 times lower values for all three binding coefficients, L1, L2, and L3 for epiregulin-EGFR binding (21). For mouse EGF binding to EGFR, we use 3 times lower values for the three binding coefficients, L1, L2, and L3 (34).

### REFERENCES

All publications, patent applications, patents, and other references mentioned in the specification and listed below are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art.
1. Schlessinger, J. Receptor tyrosine kinases: legacy of the first two decades. Cold Spring Harb. Perspect. Biol. 6, a008912 (2014).
2. Paul, M. D. & Hristova, K. The transition model of RTK activation: A quantitative framework for understanding RTK signaling and RTK modulator activity. Cytokine Growth Factor Rev. 49, 23-31 (2019).
3. Lemmon, M. A. & Schlessinger, J. Cell Signaling by Receptor Tyrosine Kinases. Cell 141, 1117-1134 (2010).
4. Karl, K. & Hristova, K. Pondering the mechanism of receptor tyrosine kinase activation: The case for ligand-specific dimer microstate ensembles. Curr. Opin. Struct. Biol. 71, 193-199 (2021).
5. Kenakin, T. Biased Receptor Signaling in Drug Discovery. Pharmacol. Rev. 71, 267-315 (2019).
6. Kenakin, T. Signaling bias in drug discovery. Expert Opin. Drug Discov. 12, 321-333 (2017).
7. Rajagopal, S. et al. Quantifying ligand bias at seven-transmembrane receptors. Mol. Pharmacol. 80, 367-377 (2011).
8. Kahsai, A. W. et al. Multiple ligand-specific conformations of the beta2-adrenergic receptor. Nat. Chem. Biol. 7, 692-700 (2011).
9. Liu, J. J., Horst, R., Katritch, V., Stevens, R. C. & Wuthrich, K. Biased signaling pathways in beta2-adrenergic receptor characterized by 19F-NMR. Science 335, 1106-1110 (2012).
10. Seyedabadi, M., Ghahremani, M. H. & Albert, P. R. Biased signaling of G protein coupled receptors (GPCRs): Molecular determinants of GPCR/transducer selectivity and therapeutic potential. Pharmacol. Ther. 200, 148-178 (2019).
11. Freed, D. M. et al. EGFR Ligands Differentially Stabilize Receptor Dimers to Specify Signaling Kinetics. Cell 171, 683-695 e618 (2017).
12. Karl, K., Paul, M. D., Pasquale, E. B. & Hristova, K. Ligand bias in receptor tyrosine kinase signaling. J. Biol. Chem. 295, 18494-18507 (2020).
13. Kuruvilla, R. et al. A neurotrophin signaling cascade coordinates sympathetic neuron development through differential control of TrkA trafficking and retrograde signaling. Cell 118, 243-255 (2004).
14. Zinkle, A. and Mohammadi, M. A threshold model for receptor tyrosine kinase signaling specificity and cell fate determination. F1000Res 7, Faculty Rev-872 (2018).
15. Kolb, P. et al. Community guidelines for GPCR ligand bias: IUPHAR review 32. Br. J. Pharmacol. 179, 3651-3674 (2022).
16. Salazar-Cavazos, E. et al. Multisite EGFR phosphorylation is regulated by adaptor protein abundances and dimer lifetimes. Mol. Biol. Cell 31, 695-708 (2020).
17. Gotoh, N. Regulation of growth factor signaling by FRS2 family docking/scaffold adaptor proteins. Cancer Sci. 99, 1319-1325 (2008).
18. Del Piccolo, N., Placone, J., He, L., Agudelo, S. C. & Hristova, K. Production of plasma membrane vesicles with chloride salts and their utility as a cell membrane mimetic for biophysical characterization of membrane protein interactions. Analy. Chem. 84, 8650-8655 (2012).
19. Sarabipour, S., Chan, R. B., Zhou, B., Di Paolo, G. & Hristova, K. Analytical characterization of plasma membrane-derived vesicles produced via osmotic and chemical vesiculation. Biochim. et Biophys. Acta 1848, 1591-1598 (2015).
20. Skinkle, A. D., Levental, K. R. & Levental, I. Cell-Derived Plasma Membrane Vesicles Are Permeable to Hydrophilic Macromolecules. Biophys. J. 118, 1292-1300 (2020).
21. Gekko, K. Solution properties of dextran and its ionic derivatives. (ACS Publications, 1981).
22. Armstrong, J. K., Wenby, R. B., Meiselman, H. J. & Fisher, T. C. The hydrodynamic radii of macromolecules and their effect on red blood cell aggregation. Biophys. J. 87, 4259-4270 (2004).
23. Jorissen, R. N. et al. Epidermal growth factor receptor: mechanisms of activation and signalling. Exp. Cell Res. 284, 31-53 (2003).
24. Abdelhamed, S., Ogura, K., Yokoyama, S., Saiki, I. & Hayakawa, Y. AKT-STAT3 Pathway as a Downstream Target of EGFR Signaling to Regulate PD-L1 Expression on NSCLC cells. J. Cancer 7, 1579-1586 (2016).
25. Sordella, R., Bell, D. W., Haber, D. A. & Settleman, J. Gefitinibsensitizing EGFR mutations in lung cancer activate anti-apoptotic pathways. Science 305, 1163-1167 (2004).
26. Byrne, P. O., Hristova, K. & Leahy, D. J. EGFR forms ligandindependent oligomers that are distinct from the active state. J. Biol. Chem. 295, 13353-13362 (2020).
27. Kenakin, T., Watson, C., Muniz-Medina, V., Christopoulos, A. & Novick, S. A simple method for quantifying functional selectivity and agonist bias. ACS Chem. Neurosci. 3, 193-203 (2012).
28. Ehlert, F. J. Analysis of Biased Agonism. Prog. Mol. Biol. Transl. Sci. 160, 63-104 (2018).
29. Shigematsu, H. & Gazdar, A. F. Somatic mutations of epidermal growth factor receptor signaling pathway in lung cancers. Int. J. Cancer 118, 257-262 (2006).
30. Black, J. W. & Leff, P. Operational models of pharmacological agonism. Proc. R Soc. Lond B Biol. Sci. 220, 141-162 (1983).
31. Nexo, E. & Hansen, H. F. Binding of epidermal growth factor from man, rat and mouse to the human epidermal growth factor receptor. Biochim. et Biophys. Acta 843, 101-106 (1985).
32. Choi, M. et al. G protein-coupled receptor kinases (GRKs) orchestrate biased agonism at the beta2-adrenergic receptor. Sci. Signal 11, eaar7084 (2018).
33. Zhou, X. E., Melcher, K. & Xu, H. E. Understanding the GPCR biased signaling through G protein and arrestin complex structures. Curr. Opin. Struct. Biol. 45, 150-159 (2017).
34. Macdonald, J. L. & Pike, L. J. Heterogeneity in EGF-binding affinities arises from negative cooperativity in an aggregating system. Proc. Natl Acad. Sci. USA 105, 112-117 (2008).
35. Tao, X., Zhao, C. & Mackinnon, R. Membrane protein isolation and structure determination in cell-derived membrane vesicles. Proc. Natl Acad. Sci. USA 120, e2302325120 (2023).
36. Chen, L. R., Novicky, L., Merzlyakov, M., Hristov, T. & Hristova, K. Measuring the Energetics of Membrane Protein Dimerization in Mammalian Membranes. J. Am. Chem. Soc. 132, 3628-3635 (2010).
37. Singh, B., Carpenter, G. and Coffey, R. J. EGF receptor ligands: recent advances. F1000Research 5, Faculty Rev-2270 (2016).
38. Krall, J. A., Beyer, E. M. & MacBeath, G. High-and low-affinity epidermal growth factor receptor-ligand interactions activate distinct signaling pathways. PloS ONE 6, e15945 (2011).
39. Sanders, J. M., Wampole, M. E., Thakur, M. L. & Wickstrom, E. Molecular determinants of epidermal growth factor binding: a molecular dynamics study. PLOS ONE 8, e54136 (2013).
40. Robertson, S. C., Tynan, J. A. & Donoghue, D. J. RTK mutations and human syndromes-when good receptors turn bad. Trends Genet. 16, 265-271 (2000).
41. Red Brewer, M. et al. Mechanism for activation of mutated epidermal growth factor receptors in lung cancer. Proc. Natl Acad. Sci. USA 110, E3595-E3604 (2013).
42. Del Piccolo, N. & Hristova, K. Quantifying the Interaction between EGFR Dimers and Grb2 in Live Cells. Biophys. J. 113, 1353-1364 (2017).
43. Sarabipour, S. & Hristova, K. FGFR3 Unliganded Dimer Stabilization by the Juxtamembrane Domain. J. Mol. Biol. 427, 1705-1714 (2015).
44. Baycin-Hizal, D. et al. Proteomic analysis of Chinese hamster ovary cells. J. Proteome Res. 11, 5265-5276 (2012).
45. Stone, M. B. & Veatch, S. L. Steady-state cross-correlations for live two-colour super-resolution localization data sets. Nat. Commun. 6, 7347 (2015).
46. Vasudevan, S. et al. Drug-Induced Resistance and Phenotypic Switch in Triple-Negative Breast Cancer Can Be Controlled via Resolution and Targeting of Individualized Signaling Signatures. Cancers (Basel) 13, 5009 (2021).
47. Yao, T. W. et al. Acquired resistance to BRAF inhibition in BRAFV600E mutant gliomas. Oncotarget 8, 583-595 (2017).
48. 48. Kenakin, T. G protein coupled receptors as allosteric proteins and the role of allosteric modulators. J. Recept. Signal Transduct. Res. 30, 313-321 (2010).
49. 49. Gundry, J., Glenn, R., Alagesan, P. & Rajagopal, S. A Practical Guide to Approaching Biased Agonism at G Protein Coupled Receptors. Front. Neurosci. 11, 17 (2017).
50. Kenakin, T. Measurement of Receptor Signaling Bias. Curr. Protoc. Pharmacol. 74, 2 15 11-12 15 15 (2016).
51. Kenakin, T. A Scale of Agonism and Allosteric Modulation for Assessment of Selectivity, Bias, and Receptor Mutation. Mol. Pharmacol. 92, 414-424 (2017).
52. Chen, L., J. Placone, L. Novicky, and K. Hristova. 2010. The extracellular domain of fibroblast growth factor receptor 3 inhibits ligand-independent dimerization. Science Signaling 3: ra86.
53. Sarabipour, S., R. B. Chan, B. Zhou, G. Di Paolo, and K. Hristova. 2015. Analytical characterization of plasma membrane-derived vesicles produced via osmotic and chemical vesiculation. Biochimica et Biophysica Acta 1848:1591-1598.
54. Chen, L. R., L. Novicky, M. Merzlyakov, T. Hristov, and K. Hristova. 2010. Measuring the Energetics of Membrane Protein Dimerization in Mammalian Membranes. Journal of the American Chemical Society 132:3628-3635.
55. Del Piccolo, N., J. Placone, and K. Hristova. 2015. Effect of Thanatophoric Dysplasia Type I Mutations on FGFR3 Dimerization. Biophysical Journal 108:272-278.
56. Otsu, N. 1979. Threshold Selection Method from Gray-Level Histograms. Ieee T Syst Man Cyb 9 (1): 62-66.
57. Jarpe, M. B., C. Knall, F. M. Mitchell, A. M. Buhl, E. Duzic, and G. L. Johnson. 1998. [D-Arg1, D-Phe5, D-Trp7, 9, Leu11] Substance P acts as a biased agonist toward neuropeptide and chemokine receptors. Journal of Biological Chemistry 273:3097-3104.
58. Michel, M. C., and S. J. Charlton. 2018. Biased agonism in drug discovery—is it too soon to choose a path? Mol Pharmacol 93:259-265.
59. Kenakin, T. 2016. Measurement of Receptor Signaling Bias. Curr Protoc Pharmacol 74:2 15 11-12 15 15.
60. Kenakin, T. 2010. G protein coupled receptors as allosteric proteins and the role of allosteric modulators. J Recept Signal Transduct Res 30:313-321.
61. Kenakin, T., and A. Christopoulos. 2013. Signalling bias in new drug discovery: detection, quantification and therapeutic impact. Nat Rev Drug Discov 12:205-216.
62. Trevor, A. J. 2015. Chapter 2. Pharmacodynamics. In Katzung & Trevor's Pharmacology: Examination & Board Review, Ile. McGraw-Hill.
63. Berg, K. A., and W. P. Clarke. 2018. Making sense of pharmacology: inverse agonism and functional selectivity. International Journal of Neuropsychopharmacology 21:962-977.
64. Sarabipour, S., and K. Hristova. 2016. Mechanism of FGF receptor dimerization and activation. Nat Commun 7:10262.
65. Huang, Z., Y. Tan, J. Gu, Y. Liu, L. Song, J. Niu, L. Zhao, L. Srinivasan, Q. Lin, J. Deng, Y. Li, D. J. Conklin, T. A. Neubert, L. Cai, X. Li, and M. Mohammadi. 2017. Uncoupling the Mitogenic and Metabolic Functions of FGF1 by Tuning FGF1-FGF Receptor Dimer Stability. Cell Rep 20:1717-1728.
66. Belliveau, D. J., I. Krivko, J. Kohn, C. Lachance, C. Pozniak, D. Rusakov, D. Kaplan, and F. D. Miller. 1997. NGF and neurotrophin-3 both activate TrkA on sympathetic neurons but differentially regulate survival and neuritogenesis. J Cell Biol 136:375-388.
67. Kuruvilla, R., L. S. Zweifel, N. O. Glebova, B. E. Lonze, G. Valdez, H. Ye, and D. D. Ginty. 2004. A neurotrophin signaling cascade coordinates sympathetic neuron development through differential control of TrkA trafficking and retrograde signaling. Cell 118:243-255.
68. Zaccaro, M. C., H. B. Lee, M. Pattarawarapan, Z. Xia, A. Caron, P.-J. L′Heureux, Y. Bengio, K. Burgess, and H. U. Saragovi. 2005. Selective small molecule peptidomimetic ligands of TrkC and TrkA receptors afford discrete or complete neurotrophic activities. Chem Biol 12:1015-1028.
69. Chen, D., F. Brahimi, Y. Angell, Y.-C. Li, J. Moscowicz, H. U. Saragovi, and K. Burgess. 2009. Bivalent peptidomimetic ligands of TrkC are biased agonists and selectively induce neuritogenesis or potentiate neurotrophin-3 trophic signals. ACS Chem Biol 4:769-781.
70. Harrington, A. W., C. St Hillaire, L. S. Zweifel, N. O. Glebova, P. Philippidou, S. Halegoua, and D. D. Ginty. 2011. Recruitment of actin modifiers to TrkA endosomes governs retrograde NGF signaling and survival. Cell 146:421-434.
71. Scarpi, D., D. Cirelli, C. Matrone, G. Castronovo, P. Rosini, E. G. Occhiato, F. Romano, L. Bartali, A. M. Clemente, G. Bottegoni, A. Cavalli, G. De Chiara, P. Bonini, P. Calissano, A. T. Palamara, E. Garaci, M. G. Torcia, A. Guarna, and F. Cozzolino. 2012. Low molecular weight, non-peptidic agonists of TrkA receptor with NGF-mimetic activity. Cell Death Dis 3: e339.
72. Freed, D. M., N. J. Bessman, A. Kiyatkin, E. Salazar-Cavazos, P. O. Byrne, J. O. Moore, C. C. Valley, K. M. Ferguson, D. J. Leahy, D. S. Lidke, and M. A. Lemmon. 2017. EGFR Ligands Differentially Stabilize Receptor Dimers to Specify Signaling Kinetics. Cell 171:683-695 e618.
73. Jorgensen, C., A. Sherman, G. I. Chen, A. Pasculescu, A. Poliakov, M. Hsiung, B. Larsen, D. G. Wilkinson, R. Linding, and T. Pawson. 2009. Cell-specific information processing in segregating populations of Eph receptor ephrin-expressing cells. Science 53. 326:1502-1509.
74. Verheyen, T., T. Fang, D. Lindenhofer, Y. Wang, K. Akopyan, A. Lindqvist, B. Högberg, and A. I. Teixeira. 2020. Spatial organization-dependent EphA2 transcriptional responses 54. revealed by ligand nanocalipers. Nucleic Acids Res 48:5777-5787.
75. Ho, C. C. M., A. Chhabra, P. Starkl, P. J. Schnorr, S. Wilmes, I. Moraga, H. S. Kwon, N. Gaudenzio, R. Sibilano, T. S. Wehrman, M. Gakovic, J. T. Sockolosky, M. R. Tiffany, A. M. Ring, J. Piehler, I. L. Weissman, S. J. Galli, J. A. Shizuru, and K. C. Garcia. 2017. Decoupling the Functional Pleiotropy of Stem Cell Factor by Tuning c-Kit Signaling. Cell 168:1041-1052 e1018.
76. Salazar-Cavazos, E., C. F. Nitta, E. D. Mitra, B. S. Wilson, K. A. Lidke, W. S. Hlavacek, and D. S. Lidke. 2020. Multisite EGFR phosphorylation is regulated by adaptor protein abundances and dimer lifetimes. Mol Biol Cell 31:695-708.
77. Kolb, P., T. Kenakin, S. P. H. Alexander, M. Bermudez, L. M. Bohn, C. S. Breinholt, M. Bouvier, S. J. Hill, E. Kostenis, K. A. Martemyanov, R. R. Neubig, H. O. Onaran, S. Rajagopal, B. L. Roth, J. Selent, A. K. Shukla, M. E. Sommer, and D. E. Gloriam. 2022. Community guidelines for GPCR ligand bias: IUPHAR review 32. Br J Pharmacol 179:3651-3674.
78. Weiss, A., and J. Schlessinger. 1998. Switching signals on or off by receptor dimerization. Cell 94:277-280.
79. Byrne, P. O., K. Hristova, and D. J. Leahy. 2020. EGFR forms ligand-independent oligomers that are distinct from the active state. The Journal of biological chemistry.
80. Needham, S. R., S. K. Roberts, A. Arkhipov, V. P. Mysore, C. J. Tynan, L. C. Zanetti-Domingues, E. T. Kim, V. Losasso, D. Korovesis, M. Hirsch, D. J. Rolfe, D. T. Clarke, M. D. Winn, A. Lajevardipour, A. H. Clayton, L. J. Pike, M. Perani, P. J. Parker, Y. Shan, D. E. Shaw, and M. L. Martin-Fernandez. 2016. EGFR oligomerization organizes kinase-active dimers into competent signalling platforms. Nat Commun 7:13307.
81. Huang, Y., S. Bharill, D. Karandur, S. M. Peterson, M. Marita, X. Shi, M. J. Kaliszewski, A. W. Smith, E. Y. Isacoff, and J. Kuriyan. 2016. Molecular basis for multimerization in the activation of the epidermal growth factor receptor. Elife 5.
82. Chung, I., R. Akita, R. Vandlen, D. Toomre, J. Schlessinger, and I. Mellman. 2010. Spatial control of EGF receptor activation by reversible dimerization on living cells. Nature 464:783-U163.
83. Macdonald, J. L., and L. J. Pike. 2008. Heterogeneity in EGF-binding affinities arises from negative cooperativity in an aggregating system. Proceedings of the National Academy of Sciences of the United States of America 105:112-117.
84. Sanders, J. M., M. E. Wampole, M. L. Thakur, and E. Wickstrom. 2013. Molecular determinants of epidermal growth factor binding: a molecular dynamics study. PLOS One 8: e54136.
85. Nexo, E., and H. F. Hansen. 1985. Binding of epidermal growth factor from man, rat and mouse to the human epidermal growth factor receptor. Biochim Biophys Acta 843:101-106.
86. Rajagopal, S., S. Ahn, D. H. Rominger, W. Gowen-MacDonald, C. M. Lam, S. M. Dewire, J. D. Violin, and R. J. Lefkowitz. 2011. Quantifying ligand bias at seven-transmembrane receptors. Mol Pharmacol 80:367-377.

What is claimed is:

1. A method for detecting receptor tyrosine kinase phosphorylation, the method comprising:
culturing cells in a medium;
transfecting the cells with nucleic acids encoding a receptor tyrosine kinase;
inducing vesiculation using osmotic stress, wherein the cells release plasma membrane-derived vesicles;
collecting the plasma membrane-derived vesicles;
adding to one or more plasma membrane-derived vesicles:
one or more ligands that bind to a receptor tyrosine kinase of a membrane of the plasma membrane-derived vesicle,
ATP kinase,
a phosphatase inhibitor, and
one or more antibodies that recognize a phosphorylated tyrosine residue on the receptor tyrosine kinase in response to ligand stimulation, wherein each antibody comprises a fluorescent label; and
detecting fluorescence of the fluorescent labels of the antibodies, thereby detecting phosphorylated tyrosine residues.

2. The method of claim 1,
wherein after vesiculation, cytoplasmic signaling proteins diffuse through the membrane of the plasma membrane-derived vesicle, and bound cytoplasmic proteins disassociate from the membrane of the plasma membrane-derived vesicle; and/or
wherein after vesiculation, cytoplasmic signaling proteins that have a hydrodynamic radii less than 4, 5, 6, 7, 8, 9, 10, 11 or 11.5 nm diffuse through the membrane of the plasma membrane-derived vesicle.

3. The method of claim 1, further comprising imaging of fluorescence intensity inside the plasma membrane-derived vesicles and fluorescence intensity outside of the plasma membrane derived-vesicles, wherein fluorescence intensity inside the plasma membrane-derived vesicles equals fluorescence intensity outside the plasma membrane-derived vesicles when phosphorylation comes to equilibrium and no feedback loop is present.

4. The method of claim 1,
further comprising allowing phosphorylation of tyrosine residues on the receptor tyrosine kinase to reach equilibrium for at least 20, 30, 40, 50, or 60 minutes prior to fluorescence imaging; and/or
further comprising quantifying phosphorylation of a tyrosine residue, wherein quantifying phosphorylation comprises imaging of fluorescence of fluorescent labels of the antibodies; and/or
further comprising labeling a receptor tyrosine kinase of a plasma membrane-derived vesicle with a fluorescent label and quantifying a receptor tyrosine kinase concentration in the membrane of the plasma membrane-derived vesicle, wherein quantifying the receptor tyrosine kinase concentration comprises imaging of fluorescence of fluorescent labels of the receptor tyrosine kinases.

5. The method of claim 1,
wherein two or more ligands that bind to a receptor tyrosine kinase are added to the one or more plasma membrane-derived vesicles; and
wherein two or more types of antibodies are added to the plasma membrane-derived vesicles, wherein a first antibody type recognizes a first phosphorylated tyrosine residue on the receptor tyrosine kinase in response to ligand stimulation and the first antibody types comprises a first fluorescent label, and wherein a second antibody type recognizes a second phosphorylated tyrosine residue on the receptor tyrosine kinase and the second antibody type comprises a second fluorescent label; and
the method further comprises screening for a ligand that induces preferential phosphorylation of one tyrosine residue on the receptor tyrosine kinase over phosphorylation of another tyrosine residue of the receptor tyrosine kinase.

6. The method of claim 1,
wherein some cells are transfected with nucleic acids encoding a wildtype receptor tyrosine kinase, and other cells are transfected with nucleic acids encoding a receptor tyrosine kinase comprising a mutation;
wherein two or more types of antibodies are added to the plasma membrane-derived vesicles collected from vesiculated cells transfected with nucleic acids encoding a wildtype receptor tyrosine kinase, wherein a first antibody type recognizes a first phosphorylated tyrosine residue on the wildtype receptor tyrosine kinase in response to ligand stimulation and the first antibody types comprises a first fluorescent label, and wherein a second antibody type recognizes a second phosphorylated tyrosine residue on the wildtype receptor tyrosine kinase and the second antibody type comprises a second fluorescent label;
wherein two or more types of antibodies are added to the plasma membrane-derived vesicles collected from vesiculated cells transfected with nucleic acids encoding a receptor tyrosine kinase comprising a pathogenic mutation, wherein a first antibody type recognizes a first phosphorylated tyrosine residue on the mutant receptor tyrosine kinase in response to ligand stimulation and the first antibody types comprises a first fluorescent label, and wherein a second antibody type recognizes a second phosphorylated tyrosine residue on the mutant receptor tyrosine kinase and the second antibody type comprises a second fluorescent label; and
the method further comprises identifying a signaling bias induced by the receptor tyrosine kinase mutation, wherein identifying the signaling bias comprises comparing i) a ligand's induced preferential phosphorylation of one tyrosine residue of the wildtype receptor tyrosine kinase over phosphorylation of another tyrosine residue of the wildtype receptor tyrosine kinase and ii) the same ligand's induced preferential phosphorylation of one tyrosine residue of the mutant receptor tyrosine kinase over phosphorylation of another tyrosine residue of the mutant receptor tyrosine kinase.

7. The method of claim 6, wherein the mutation is a pathogenic mutation.

8. The method of claim 1, wherein each ligand comprises a fluorescent label; and the method further comprises quantifying ligand binding, wherein quantifying ligand binding comprises imaging of fluorescence of fluorescent labels of ligands.

9. The method of claim 8, further comprising quantifying receptor tyrosine kinase phosphorylation upon ligand stimulation for the plasma membrane-derived vesicle, wherein quantifying receptor tyrosine kinase phosphorylation upon ligand stimulation comprises quantifying ligand binding to the receptor tyrosine kinase and quantifying phosphorylation of a tyrosine residue simultaneously.

10. The method of claim 9,
wherein quantifying ligand binding to the receptor tyrosine kinase comprises imaging fluorescence of the fluorescent labels of the ligands on the membrane of a plasma membrane-derived vesicle, and
wherein quantifying phosphorylation of a tyrosine residue comprises imaging fluorescence of the fluorescent labels of the antibodies.

11. The method of claim 10, further comprising
labeling a receptor tyrosine kinase of a plasma membrane-derived vesicle with a fluorescent label and quantifying a receptor tyrosine kinase concentration in the membrane of the plasma membrane-derived vesicle, wherein quantifying the receptor tyrosine kinase concentration comprises imaging of fluorescence of fluorescent labels of the receptor tyrosine kinases; and
quantifying a maximum phosphorylation per receptor in response to one ligand for a maximum of ligand-bound receptors per plasma membrane-derived vesicle.

12. The method of claim 1, further comprising quantifying recruitment of antibodies to the vesicle membrane, wherein quantifying recruitment of antibodies to the vesicle membrane comprises imaging of fluorescent membrane intensities.

13. The method of claim 1, wherein the ligands comprise EGF, TGFα, Epiregulin, or EGF-tetramethylrhodamine.

14. The method of claim 1, wherein the nucleic acids are plasmid DNA encoding the extracellular and transmembrane domain (ECTM) of FGFR.

15. The method of claim 1, wherein the nucleic acids are plasmid DNA encoding human epidermal growth factor receptor (EGFR).

16. The method of claim 1, wherein the nucleic acids comprise a fluorescent label.

17. The method of claim 15, wherein the cultured cells do not exhibit endogenous EGFR.

18. The method of claim 1, wherein the antibodies have a hydrodynamic radii less than 4, 5, 6, 7, 8, 9, 10, 11 or 11.5 nm.

19. The method of claim 1, wherein data processing of images is automated.

20. The method of claim 19, wherein data processing of images is automated using a neural network.


SEQUENCE LISTING (2 sequences)

SEQ ID NO: 1 - DNA, 31 bp, Synthetic construct
  source 1..31: mol_type = other DNA
cccagcagtttggcccgcccaaaatctgtga

SEQ ID NO: 2 - DNA, 31 bp, Synthetic construct
  source 1..31: mol_type = other DNA
tcacagattttgggcgggccaaactgctggg